(19) United States
(12) Patent Application Publication
(10) Pub. No.: US 2025/0025560 A1
(43) Pub. Date: Jan. 23, 2025

(54) METHOD FOR THE REVIVAL OF MICROGLIA AND ASTROCYTES AND APPLICATIONS THEREOF

(71) Applicant: UNIVERSITY OF SOUTH CAROLINA, Columbia, SC (US)

(72) Inventors: Peisheng Xu, CHAPIN, SC (US); Mingming Wang, WEST COLUMBIA, SC (US)

(21) Appl. No.: 18/648,946

(22) Filed: Apr. 29, 2024

Related U.S. Application Data
(60) Provisional application No. 63/589,727, filed on Oct. 12, 2023.
(60) Provisional application No. 63/512,934, filed on Jul. 11, 2023.

(51) Int. Cl. A61K 47/34; A61K 39/00; A61K 47/69; A61P 25/28; C07K 16/28

(52) U.S. Cl. CPC A61K 47/34; A61K 47/6903; A61P 25/28; C07K 16/2827; A61K 2039/505

(57) ABSTRACT

Methods are described for treatment of Alzheimer's Disease as well as other neurodegenerative disorders in which microglia and/or astrocyte abilities have been altered and impaired. Methods include brain-targeting of PD-L1 using a brain targeted nanogel loaded with PDL1 antibody and functionalized with two particular targeting ligands. The nanogels can successfully cross the blood brain barrier to release the antibody and reduce PD-L1 expression in the brain while preserving the integrity of the BBB.

### CROSS REFERENCE TO RELATED APPLICATION

This application claims filing benefit of U.S. Provisional Patent Application Ser. No. 63/512,934 having a filing date of Jul. 11, 2023, entitled “Method for the Revival of Microglia and Astrocytes and its Application,” and of U.S. Provisional Patent Application Ser. No. 63/589,727 having a filing date of Oct. 12, 2023, entitled “Method for the Revival of Microglia and Astrocytes and Applications Thereof,” both which being incorporated herein by reference for all purposes.

### FEDERAL RESEARCH STATEMENT

This invention was made with Government support under Grant No. 1R01AG054839-01A1, awarded by the National Institutes of Health. The Government has certain rights in the invention.

### BACKGROUND

Alzheimer's disease (AD) is the sixth-leading cause of death in the United States. The development and progression of AD are the collective consequence of the toxicities and inflammatory microenvironment induced by β-amyloid (Aβ) plaques, tau protein-formed neurofibrillary tangles, and malfunction of microglia. Thus far, most approaches explored for the treatment of AD have been focused on reducing the amount of toxic substances and attenuating the inflammatory status of the brain to protect neurons from damage. However, due to the limitation of those approaches, both in their targets and efficacies, currently available approaches for AD therapy show only limited efficacy.

Despite many failures in targeting Aβ for AD therapy, recently, several Aβ targeted antibodies, including lecanemab and aducanumab, yielded plausible results in clinical trials, such as reduced Aβ burden and slowed progression of the disease. Unfortunately, none of them managed to stop AD progression due to the existence of the blood-brain barrier (BBB), which prevents 98% of small molecules and almost all macromolecules from entering the brain.

Microglia, the resident macrophages of the brain, are continually surveying the brain for the incidence of pathogens and cell debris and providing maintenance for the brain tissue. At an early stage of AD, activated microglia engulf Aβ fibrils by phagocytosis. With the progression of the disease combined with aging, the phagocytic abilities of microglia are altered and impaired, causing the accumulation of Aβ fibrils in the brain, resulting in super-activation of microglial cells and promoting inflammatory responses to produce neuronal toxic reactive oxygen species and pro-inflammatory cytokines. Consequently, the damaged neurons send neuronal injury signals to microglia to further activate microglial cells and propagate the cycle. The continuation of this progressive cycle causes neuron loss and AD progression. In addition, studies have revealed that the activation of astrocytes is closely related to progression of AD, including failure to clear Aβ fibrils and microglial debris, unleashing Aβ effects in pathological tau phosphorylation, aberrantly producing γ-aminobutyric acid to inhibit neighboring neuronal activity and causing neuronal dysfunction in AD.

Dysregulation of programmed death ligand 1 (PD-L1), which is expressed in both microglia and astrocytes, plays a pivotal role in modulating the immune response in Alzheimer's disease (AD) and other central nervous system (CNS) diseases. This dysregulation hinders efficient amyloid-beta (Aβ) plaque clearance and leads to detrimental microglial activation. It has been previously reported that the expression of PD-L1 in microglia and astrocytes is upregulated in both AD patients and AD mouse models. It has also been reported that PD-L1 knockout mice had boosted M2 microglia, which featured anti-inflammatory and neuroprotective effects.

PD-L1 antibody (aPDL1) has been extensively adopted in cancer immunotherapy by blocking the interaction between PD-1 and PD-L1. Studies have explored the efficacy of aPDL1 in mouse AD models, from which it has been discovered that aPDL1 can attenuate AD related symptoms, including reduced Aβ fibrils burden and tau aggregation in 5XFAD and DM-hTAU models, respectively, as well as attenuating inflammatory cytokines and alleviating cognitive deficits for both models. Unfortunately, due to the existence of the blood-brain barrier (BBB), only a limited fraction of aPDL1 can reach the brain region after i.v. administration, which makes its clinical application impractical.

The BBB, formed from the adherents and tight junctions of endothelial cells, protects the brain from damage and bacterial infection by limiting the passage of high molecular weight molecules and pathogens. However, due to the presence of the BBB, more than 98% of existing therapeutic molecules cannot enter the brain. Consequently, therapeutics for central nervous system (CNS) diseases cannot reach their targets efficiently and thus fail to exhibit therapeutic effects. Various carrier systems have been developed in at attempt to deliver therapeutics across the BBB, including polymers, dendrimers, nanogels, mesoporous silica particles, and gold nanoparticles.

While such development has provided improvement in the art, room for further improvement exists. What is needed in the art is a method for treatment of a degenerative disorder such as AD that provides for successful delivery of aPD-L1 across the BBB and thereby revive beneficial activities of microglia and astrocytes.

### SUMMARY

According to one embodiment, disclosed is a method for reviving function in impaired microglia and/or astrocytes. For instance, a method can include delivering a nanogel to the microglia and/or astrocytes. The nanogel includes a crosslinked network that in turn includes a first copolymer and a second copolymer. The first copolymer includes a backbone, a first group pendant to the first backbone that includes a poly(ethylene glycol), and a second group pendant to the backbone that is conjugated to a PD-L1 antibody via a disulfide bond. The second copolymer also includes a second backbone, with a group pendant to the second backbone that includes a poly(ethylene glycol), and another group pendant to the backbone of the second copolymer that is conjugated to a nictotinic acetylcholine receptor (nAChR) targeting ligand via a disulfide bond. The nanogel also includes a targeting ligand for receptor for advanced glycation endproducts (RAGE), with this targeting ligand conjugated at a surface of the nanogel via a disulfide bond. According to the method, following delivery of the nanogel to the impaired microglia and/or astrocytes, the various disulfide bonds are degraded, thereby releasing the PD-L1 antibody. Following release of the PD-L1 antibody, PD-L1 expressed by the microglia and/or astrocytes is degraded and the function of the impaired microglia and/or astrocytes is revived.

Also disclosed are methods for treatment of neurodegenerative disorders. A treatment method can generally include delivering a pharmaceutical composition to a subject suffering from the neurodegenerative disorder, the pharmaceutical composition including a nanogel as described and a pharmaceutically effective carrier. Upon delivery of the pharmaceutical composition to the subject, the nanogel can cross the blood brain barrier, following which the various disulfide bonds are degraded, thereby releasing the PD-L1 antibody, leading to degradation of PD-L1 expressed by impaired microglia and/or astrocytes in the brain and revival of function of impaired microglia and/or astrocytes.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a scheme for revival of microglia and astrocytes by use of disclosed materials for AD therapy.

FIG. 2 presents a reaction scheme for formation of poly [(2-(pyridin-2-yldisulfanyl)ethyl acrylate)-co-[poly(ethylene glycol)methoxy]](PDA-PEG-OCH₃).

FIG. 3 presents a reaction scheme for formation of poly [(2-(pyridin-2-yldisulfanyl)ethyl acrylate)-co-[poly(ethylene glycol)hydroxy]](PDA-PEG-OH).

FIG. 4 presents a reaction scheme for formation of poly [(2-(pyridin-2-yldisulfanyl)ethyl acrylate)-co-[poly(ethylene glycol)[-co-[poly(2-mercaptoethanol)]](PDA-PEG-BME) intermediate copolymer.

FIG. 5 presents a reaction scheme for formation of poly [(2-(pyridin-2-yldisulfanyl)ethyl acrylate)-co-[poly(ethylene glycol)[-co-[poly(4-nitrophynyl chloroformate)]](PDA-PEG-NPC) precursor copolymer.

FIG. 6 presents a reaction scheme for formation of poly [(2-(pyridin-2-yldisulfanyl)ethyl acrylate)-co-[poly(ethylene glycol)]-co-[poly(glutathione-2-methacryloyloxyethyl phosphorylcholine)]](PDA-PEG-MPC) copolymer.

FIG. 7 presents a reaction scheme for formation of a PDA-PEG-antibody conjugate copolymer.

FIG. 8 presents a reaction scheme for formation of a PDA-PEG-MPC-antibody nanogel through crosslinking of the PDA-PEG-MPC copolymer with the PDA-PEG-antibody conjugate copolymer.

FIG. 9 presents a reaction scheme for formation of a dual targeted nanogel through surface decoration of a nanogel with a ligand for RAGE.

FIG. 10 illustrates a formation approach for forming a brain targeted nanogel as described in the Examples section.

FIG. 11 provides a PAGE gel electrophoresis image including nanogels (BTN-PLD1) as described herein.

FIG. 12 provides a TEM image of nanogels as described herein.

FIG. 13 presents a size distribution of exemplary nanogels as described herein.

FIG. 14 illustrates the flow cytometry spectra for cellular uptake and lysosomal escape of nanogels as described herein (BTN-PDL1) with hCMEC/d3 cells after 4 hours co-culture.

FIG. 15 illustrates the quantitative analysis for cellular uptake and lysosomal escape of nanogels as described herein (BTN-PDL1) with hCMEC/d3 cells after 4 hours co-culture.

FIG. 16 illustrates the flow cytometry spectra for cellular uptake and lysosomal escape of nanogels as described herein (BTN-PDL1) with BV2 cells after 4 hours co-culture.

FIG. 17 illustrates the quantitative analysis for cellular uptake and lysosomal escape of nanogels as described herein (BTN-PDL1) with BV2 cells after 4 hours co-culture.

FIG. 18 illustrates the flow cytometry spectra for cellular uptake and lysosomal escape of nanogels as described herein (BTN-PDL1) with mouse astrocyte (MA) cells after 4 hours co-culture.

FIG. 19 illustrates the quantitative analysis for cellular uptake and lysosomal escape of nanogels as described herein (BTN-PDL1) with MA cells after 4 hours co-culture.

FIG. 20 schematically illustrates a BBB transwell model used in the examples section.

FIG. 21 presents the penetration efficiency of BSA-Cy3 and BTN-BSACy3 assessed under various conditions in the transwell model of FIG. 20.

FIG. 22 presents immunofluorescence images of in vivo biodistribution of nanogels (BTN-PDL1) in 5xFAD mice.

FIG. 23 presents the ex vivo biodistribution of nanogels (BTN-PDL1) in the brains of 5xFAD mice.

FIG. 24 illustrates the expression of TRIM21 in BV2 and MA cells.

FIG. 25 illustrates via gel electrophoresis the expressions of PD-L1 in BV2 cells.

FIG. 26 quantitatively illustrates the expression of PD-L1 in BV2 cells.

FIG. 27 illustrates via gel electrophoresis the expressions of PD-L1 in MA cells.

FIG. 28 quantitatively illustrates the expression of PDL-1 in BV2 cells.

FIG. 29 illustrates via gel electrophoresis the expression of ZO-1 and Occludin in hCME/D3 cells.

FIG. 30 quantitatively illustrates the expression of ZO-1 in hCMEC/D3 cells.

FIG. 31 quantitatively illustrates the expression of Occludin in hCMEC/D3 cells.

FIG. 32 illustrates the cell viability of BV2 cells upon the challenge of 100 ng/mL LPS.

FIG. 33 illustrates the cell viability of N2a converted neurons after being incubated with CMBV-2+LPS for 48 hours.

FIG. 34 quantitatively illustrates the remaining Aβ-FITC in a system including nanogels (BTN-PDL1) or aPDL1 co-cultured with BV2 cells and 5 μM Aβ-FITC.

FIG. 35 schematically illustrates a transwell model used in Examples set forth herein.

FIG. 36 quantitatively illustrates the remaining Aβ in the system of FIG. 35 loaded with nanogels as described, Cy5-labeled BV2 cells, Aβ-FITC, and N2a converted neurons after 24 hours of treatment.

FIG. 37 quantitatively illustrates the infiltrated BV2 cells in the system of FIG. 35 loaded with nanogels as described, Cy5-labeled BV2 cells, Aβ-FITC, and N2a converted neurons after 24 hours of treatment.

FIG. 38 quantitatively illustrates the remaining neurons in the system of FIG. 35 loaded with nanogels as described, Cy5-labeled BV2 cells, Aβ-FITC, and N2a converted neurons after 24 hours of treatment.

FIG. 39 quantitatively illustrates amount of remaining BV2 cell debris following 96 hours of co-culture of MA cells fed with Cy5-labeled cell debris of BV2 and Aβ-FITC.

FIG. 40 quantitatively illustrates amount of remaining Aβ following 96 hours of co-culture of MA cells fed with Cy5-labeled cell debris of BV2 and Aβ-FITC.

FIG. 41 presents representative photos of nest-building in an in vivo treatment regime for a mouse model.

FIG. 42 quantitatively illustrates the nesting scores in different groups in an in vivo treatment regime for a mouse model.

FIG. 43 presents representative swimming paths in an in vivo treatment regime for a mouse model.

FIG. 44 quantitatively illustrates the escape latency in different groups in an in vivo treatment regime for a mouse model.

FIG. 45 quantitatively illustrates the times of crossing an escape platform zone in an in vivo treatment regime for a mouse model.

FIG. 46 illustrates PD-L1 expression assessed using whole brain immunoblotting.

FIG. 47 quantitatively illustrates whole brain PD-L1 expression values.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Disclosed herein is a method for treatment of AD and other neurodegenerative disorders in which microglia and/or astrocyte abilities have been altered and impaired. More specifically, disclosed is a method of treatment that includes therapeutic brain-targeting of PD-L1 using a brain targeted nanogel (BTN) loaded with aPDL1 and functionalized with two particular targeting ligands. Upon delivery of aPDL1 across the BBB according to disclosed methods, the antibody can be released from the nanogel and reduce PD-L1 expression in the brain while preserving the integrity of the BBB, and thereby promote the revival of desirable activities and cooperative interactions between microglia and astrocytes, facilitating the degradation of Aβ plaques and enhancing neuronal survival and cognitive function. The therapeutic targeting of PD-L1 with aPDL1 as disclosed herein can present a promising avenue to revival of microglia and astrocytes, improving neuronal health and cognitive function in AD as well as other central nervous system (CNS) diseases.

More specifically, disclosed are therapy applications using a brain-targeted nanogel (also referred to throughout this disclosure as BTN-PDL1) that integrates the function of ligands targeting two particular receptor types, nAChR and RAGE, with aPDL1 to deliver the nanogel across the BBB and subsequently degrade disulfide bonds of the nanogels in the brain environment, thereby releasing the antibody, which in turn downregulates PD-L1 in the microglia and astrocytes and revive their function in clearing Aβ and attenuating neuroinflammation.

Without wishing to be bound to any particular theory, disclosed methods are understood to function via an endogenous protein degradation mechanism that can selectively degrade proteins in mammalian cells by intracellularly delivering antibodies. The endogenous methodology utilizes an intrinsic cellular self-defense machinery, which involves the intracellular supplying of a PDL1 antibody, binding of the antibody to its target protein PDL1, and formation of an antibody/target protein complex with tripartite motif containing-21 (TRIM21), followed by ubiquitination and proteasome-mediated degradation of the complex.

The first of the two receptor types targeted by the nanogels are nicotinic acetylcholine receptors (nAchR). nAchR are ligand-gated cation channels located on presynaptic compartment sites where they modulate acetylcholine or other neurotransmitter release, as well as cell excitability and neuronal integration. nAchR expression is extremely broad across the brain.

The second of the two receptor types targeted by the nanogels are receptors for advanced glycation endproduces (RAGE). During neuroinflammation, RAGE is overexpressed on the BBB. Furthermore, RAGE is highly expressed in the brain of AD patients. As such, delivery materials for use in disclosed methods beneficially include a ligand that targets RAGE.

Through incorporation of the two different types of targeting ligands in conjunction with aPDL1 with a nanogel designed to degrade in the relatively high acidic and high redox potential environment of the brain, disclosed materials can be utilized in therapeutic approaches to deliver the materials across the BBB and to the targeted cell types so as to release the payload in targeted areas and thereby help stop the progression of disease states such as AD and provide a route to improved retention of learning and cognitive capacity in subjects treated according to disclosed methods.

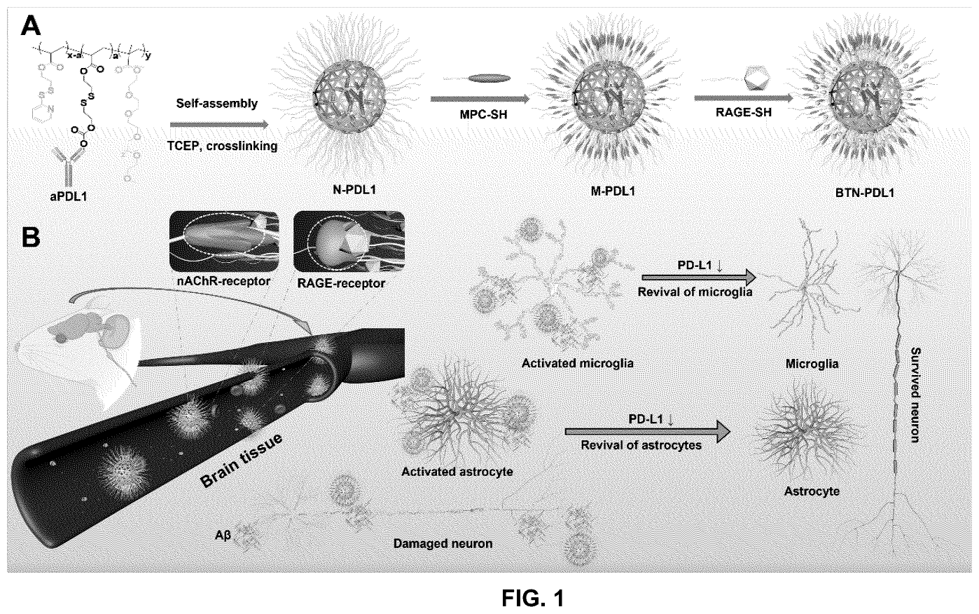

FIG. 1 schematically describes methods for forming and utilizing disclosed materials. At A of FIG. 1 is illustrated a fabrication scheme of the brain targeted materials. At B of FIG. 1 is schematically illustrated a method of action including materials crossing the BBB through RAGE and nAChR receptor-mediated transcytosis, beyond which the delivery vehicle can enter microglia and astrocytes and thereafter release the aPDL1 payload upon degradation of disulfide bonds of the nanogels and subsequently degrade intracellular PD-L1. Through degradation of intracellular PD-L1, both microglia and astrocytes can be revived for proper function. For instance, the revived cells can clear Aβ plaques and protect neurons and the BBB from Aβ induced toxicity.

The brain targeted system can be used for the treatment of any of a variety of neurodegenerative diseases including, without limitation, Alzheimer's disease, Amyotrophic lateral sclerosis, Friedreich's ataxia, Huntington's disease, Lewy body disease, Parkinson's disease, spinal muscular atrophy, multiple sclerosis, neonatal hypoxic-ischemic, stroke, spinal cord injury, brain injury, retina injury, post-traumatic stress disorder, brain tumor, glioblastroma, chemobrain, long COVID, and frontotemporal dementia, among others.

The delivery system is in the form of nanogel particles that include crosslinked copolymers that provide the nanoparticle structure of the delivery system, as well as that carry the aPD-LI antibody and the targeting ligands by use of disulfide-bond containing pendant ligands of the nanogels. For instance, copolymers of a crosslinked nanogel can be conjugated with one or more of an antibody, a ligand for a targeted receptor, as well as one or more additional desirable components such as additional ligands for targeting a particular cellular component.

As utilized herein, the term “nanogel” refers to a particle having an average cross-sectional dimension on the nanoscale, e.g., less than 1000 nanometers, such as about 500 nanometers or less, about 300 nanometers or less, about 100 nanometers or less, about 50 nanometers or less, about 30 nanometers or less, about 20 nanometers, or about 10 nanometers or less, such as from about 5 nanometer to about 50 nanometers in some embodiments. A nanogel particle can also be highly hydrophilic and capable of high water content.

A copolymer of a crosslinked nanogel can be developed from an initial reaction product of a poly(ethylene glycol) and a pyridine-2-thiol-containing monomer. A copolymer can be a block copolymer, a random copolymer, or any combination thereof. While illustrated in the present disclosure as block copolymers, it will be understood that this representation is simply shorthand for any type of copolymer (e.g., random, block, etc.) that includes repeating units, including pendant groups as described herein.

A copolymer formed upon reaction of a poly(ethylene glycol) and a pyridine-2-thiol-containing monomer can include first groups pendant to the polymer backbone that include hydrophilic poly(ethylene glycol), as well as second groups pendant to the polymer backbone that are pyridine-terminated and that also include a disulfide linkage between the polymer backbone and the terminal pyridine group. Pyridine groups of a copolymer thus formed can be further reacted in one or more intermediate formation steps to conjugate the aPDL1 and the two BBB targeting ligands as well as any other desired component of a delivery system to the copolymer and form copolymers that can also be crosslinked to form a nanogel.

For instance, a first copolymer can include an aPDL1 conjugated to a pendant group and a second copolymer can include a phosphorylcholine group as a component of the pendant group (e.g., a terminal component of a pendant group), and both copolymers can include hydrophilic poly(ethylene glycol) pendant groups as well as unreacted groups for further conjugation.

The antibody and targeting ligands can be bonded to the nanoparticle via disulfide-containing linkages that can degrade in an environment including acidic pH and/or high redox potential. As such, the nanoparticles can circulate in a subject's system, pass the BBB through the targeting provided by BBB receptor targeting ligands, and then release the aPDL1 payload due to the sensitivity of the disulfide ligand attachment bonds to the environment of the blood brain barrier lysosome and/or the nervous system.

The nanoparticles can be prepared from polymeric materials that can be biocompatible, provide long circulation life in a body, and that can be successfully ligated to at least two different ligands via an acidic responsive and/or redox potential-responsive bond formation. As utilized herein, an acidic-sensitive bond can generally refer to a bond that will degrade or otherwise break in an environment of about pH 6.8 or less, for instance about pH 4 to about pH 6.8, and will be more stable in an environment at higher pH (e.g., about 7 or higher). A redox potential-sensitive bond can generally refer to a bond that will degrade in an environment having a redox potential equal to that of a glutathione concentration of from about 0.1 mM to about 10 mM).

Upon crosslinking among and between the two (or more) copolymers, e.g., a crosslinking reaction in which disulfide bonds of unreacted pendant groups of the copolymers are cleaved followed by aerial oxidation, a resulting nanogel particle can be formed that can encapsulate the antibody as well as display hydrophilic poly(ethylene glycol) and other desirable groups at the surface and/or encapsulated within the nanogel. The ligands for the targeted receptors can be conjugated to a copolymer either prior to or following crosslinking so as to be present on the external surface of the crosslinked nanogel and encourage transport of the nanogel across the BBB as well as targeting microglia and/or astrocytes within the brain. For instance, one or both of the ligands can be conjugated to remaining reactive groups of one or more of the copolymers following crosslinking and nanogel formation. Alternatively, one or both of the ligands can be conjugated to reactive groups of one or more of the copolymers prior to crosslinking and nanogel formation.

The nanogel particles thus formed can include a hydrophilic poly(ethylene glycol) component, with both targeting ligands and aPDL1 for therapeutic delivery conjugated to the nanogel copolymers via acid-sensitive disulfide bonds. The targeting ligands can be present in high concentration at the exterior surface of the nanogel particles. The formation of the nanogel particles can endow advantages for central nervous system therapy. For example, due to the existence of the hydrophilic poly(ethylene glycol) pendant groups, the circulation time of the nanogels in a systemic delivery approach can be greatly extended. Moreover, the aPDL1 can be encapsulated within the crosslinked nanogels and thus protected from degradation prior to delivery from the nanogel following crossing of the BBB. As the copolymers forming the nanogels and conjugating the antibody to the nanogels can include linkages sensitive to an environment common on the brain side of the BBB barrier, the aPDL1 can be released from the nanogels upon passage of the nanogel into an environment conducive to linkage degradation, e.g., following crossing of the BBB by the nanogel and in some embodiments following uptake by cells on the brain side of the BBB.

A poly(ethylene glycol) used in forming a delivery system can include reactivity for reaction with a pyridine-2-thiol monomer, e.g., a methacrylate functionality. In one particular embodiment, an initial copolymer formation scheme can include reaction of pyridine-2-thiol monomer with poly(ethylene glycol) methacrylate having the general structure:

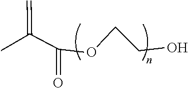

in which n is from about 4 to about 1,000, from about 5 to about 100, or from about 6 to about 20 in some embodiments.

A poly(ethylene glycol) methacrylate is not limited to the above, however, and can include modification at a terminal hydroxyl group. For instance, the poly(ethylene glycol) starting material can include, without limitation, poly(ethylene glycol) methacrylate, poly(ethylene glycol) methyl ether methacrylate, etc.

A pyridine-2-thiol-containing monomer can be copolymerized with a poly(ethylene glycol) to form a copolymer that includes the pyridine-2-thiol groups pendant to the copolymer backbone. By way of example, and without limitation, pyridine-2-thiol monomers can include one or more of:

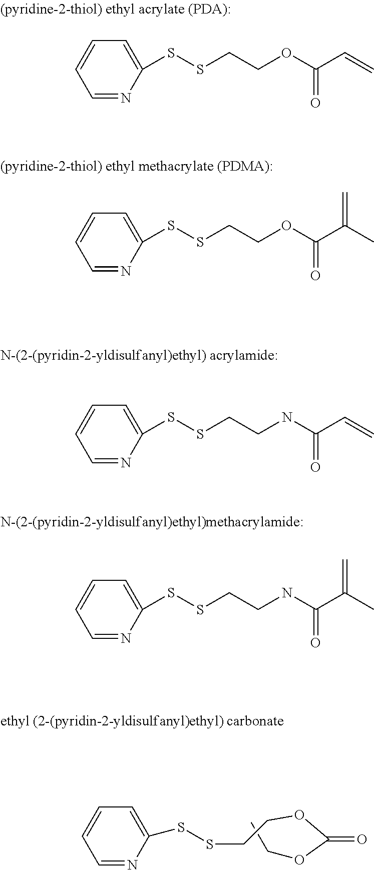 [SMILES: C=CC(=O)OCCSSc1ccccn1 ; C=C(C)C(=O)OCCSSc1ccccn1 ; C=CC(=O)NCCSSc1ccccn1 ; C=C(C)C(=O)NCCSSc1ccccn1 ; CCOC(=O)OCCSSc1ccccn1]

The reaction can be facilitated by any suitable catalyst. For example, a catalyst can include, without limitation, azobisisobutyronitrile (AIBN), benzoyl peroxide, potassium persulfate, or combinations thereof. The polymerization can be free radical polymerization or living radical polymerization including stable free radical mediated polymerization (SFRP), atom transfer radical polymerization (ATRP), reversible addition-fragmentation chain transfer (RAFT) polymerization, and iodine-transfer polymerization. The last monomer of the above examples (ethyl(2-(pyridin-2-yldisulfanyl)ethyl) carbonate) can be polymerized using isopropanol as an initiator and Sn(Oct)₂ as a catalyst through ring-opening polymerization.

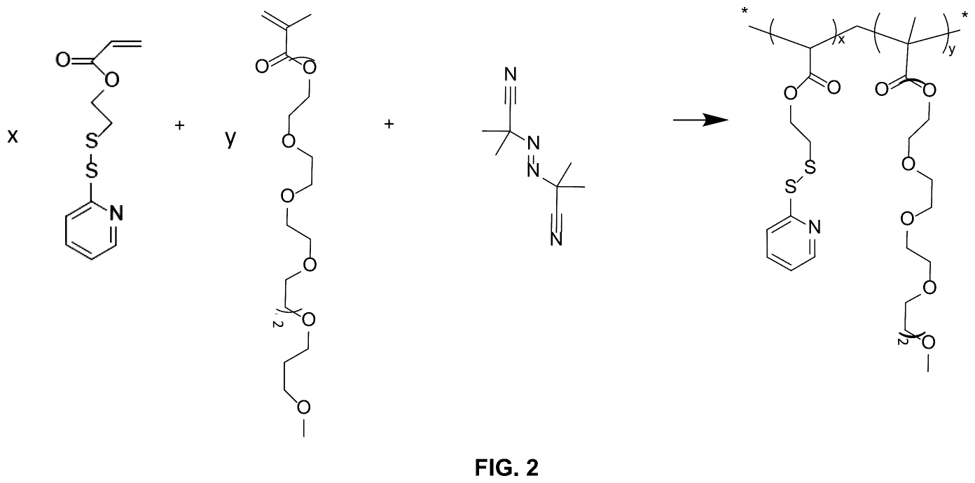

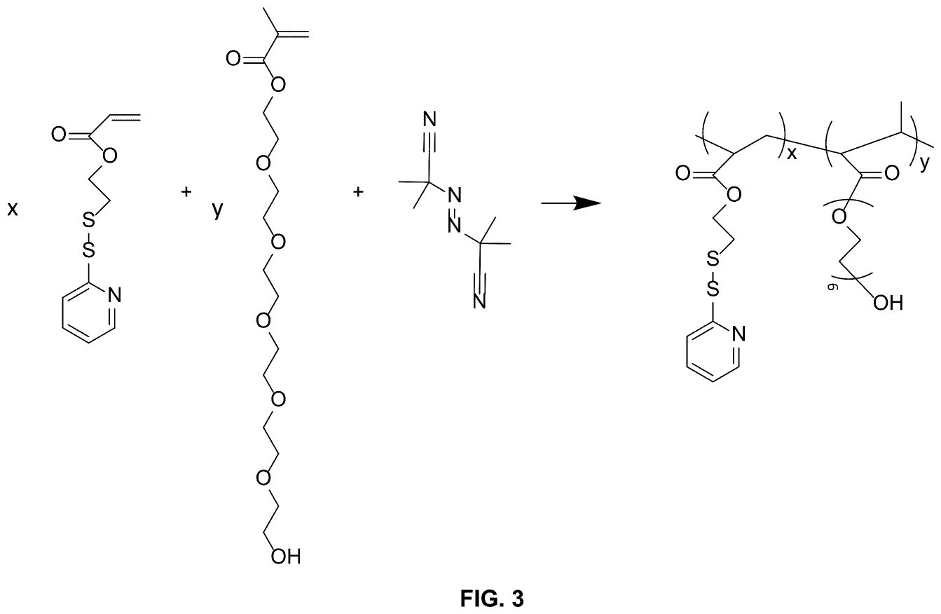

By way of example, FIG. 2 illustrates a reaction scheme of a 2-(pyridin-2-yldisulfanyl)ethyl acrylate (PDSA) monomer with poly(ethylene glycol) methyl ether methacrylate (PEGMMA), and FIG. 3 illustrates a reaction scheme of PDSA with poly(ethylene glycol) methacrylate (PGMA), both using azobisisobutyronitrile (AIBN) as catalyst. As can be seen, the poly(ethylene glycol) component of the copolymers thus formed can be in the form of groups pendant to the copolymer backbone, with second pendant groups including a disulfide linkage and an ester linkage and terminating in a pyridine group.

While illustrated with particular chain lengths, it should be understood that the figures illustrate representative embodiments only, and the length of the poly(ethylene glycol) pendant units as well as the molar ratio of the pyridine-2-thiol-containing repeating units of the polymer to the poly(ethylene glycol) pendant units is not particularly limited. For instance, the molar ratio of the pyridine-2-thiol-containing repeating units of the polymer to the poly(ethylene glycol) pendant units can be from about 100:1 to about 1:100 (the ratio of x: y in FIG. 1 and FIG. 2), for instance, from about 20:1 to about 1:20 in some embodiments, from about 10:1 to about 1:10 in some embodiments, or about 1:1 in some embodiments.

A formed copolymer can generally have a weight average molecular weight from about 1,000 to about 100,000 or from about 5,000 to about 35,000 in some embodiments. In one embodiment, the copolymer can have a polydispersity index (PDI) of from about 1.05 to about 3 or from about 1.15 to about 1.30, in some embodiments.

The PDI is a measure of the distribution of molecular mass in a given polymer sample. The PDI calculated is the weight average molecular weight divided by the number average molecular weight. It indicates the distribution of individual molecular masses in a batch of polymers. The PDI has a value equal to or greater than 1, but as the polymer chains approach uniform chain length, the PDI approaches unity (i.e., 1).

The number average molecular weight (Mₙ) is readily calculated by one of ordinary skill in the art, and generally refers to the ordinary arithmetic mean or average of the molecular weights of the individual macromolecules. It is determined by measuring the molecular weight of n polymer molecules, summing the weights, and dividing by n, such as represented in the formula:

$${\overline{M}}_{n} = \frac{{\sum }_{i} {N}_{i} {M}_{i}}{{\sum }_{i} {N}_{i}}$$

where Nᵢ is the number of molecules of molecular weight Mᵢ. The number average molecular weight of a polymer can be determined by gel permeation chromatography, and all colligative methods, like vapor pressure osmometry or end-group determination.

The weight average molecular weight (M_{w}) is readily calculated by one of ordinary skill in the art, and generally refers to:

$${\overline{M}}_{w} = \frac{{\sum }_{i} {N}_{i} {M}_{i}^{2}}{{\sum }_{i} {N}_{i} {M}_{i}}$$

where Nᵢ is the number of molecules of molecular weight Mᵢ as above. The weight average molecular weight can be determined by light scattering, small angle neutron scattering (SANS), X-ray scattering, gel permeation chromatography, and sedimentation velocity.

A copolymer formed upon reaction of a poly(ethylene glycol) with a pyridine-2-thiol-containing monomer, e.g., poly [(2-(pyridin-2-yldisulfanyl)ethyl acrylate-co-[poly(ethylene glycol) (PDA-PEG), can be a precursor copolymer that can be further reacted directly with a compound or monomer containing a functional group of use in the final delivery system (e.g., the aPDL1 or one of the BBB receptor ligands) to form a copolymer that is then crosslinked to form a nanogel. In some embodiments, a copolymer formed upon initial reaction of a poly(ethylene glycol) with a pyridine-2-thiol-containing monomer can be an intermediate copolymer that can then be further reacted to form one or more further intermediates and/or a precursor copolymer that can then be reacted directly with a material for use in the final delivery system and thereby form a copolymer that is then crosslinked to form a nanogel. In general, further functionalization of a precursor or intermediate copolymer can be carried out via reaction of pyridine groups (or reaction products of pyridine groups) of the copolymer.

To conjugate the aPD-L1 antibody to a precursor copolymer, pyridine groups of an intermediate copolymer can be activated with a functional group that is conducive to antibody conjugation. For instance, an intermediate copolymer can be functionalized by reaction with an activator having a general structure of Y-Q-X. X and Y independently may be a leaving group, one of which (Y) being capable of reacting with a pyridine group or an intermediary reaction product group provided upon reaction of a monomer with a pyridine group and the other (X) being capable of conjugation with an antibody.

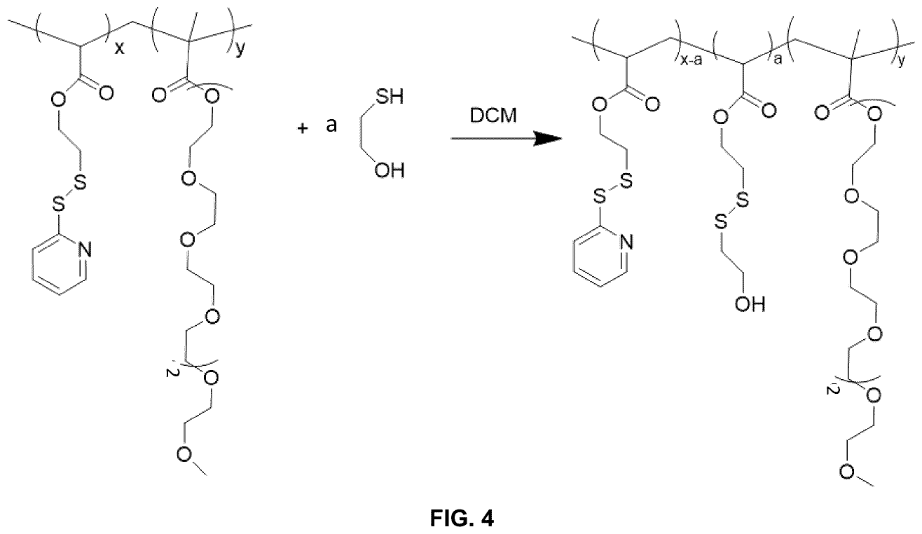

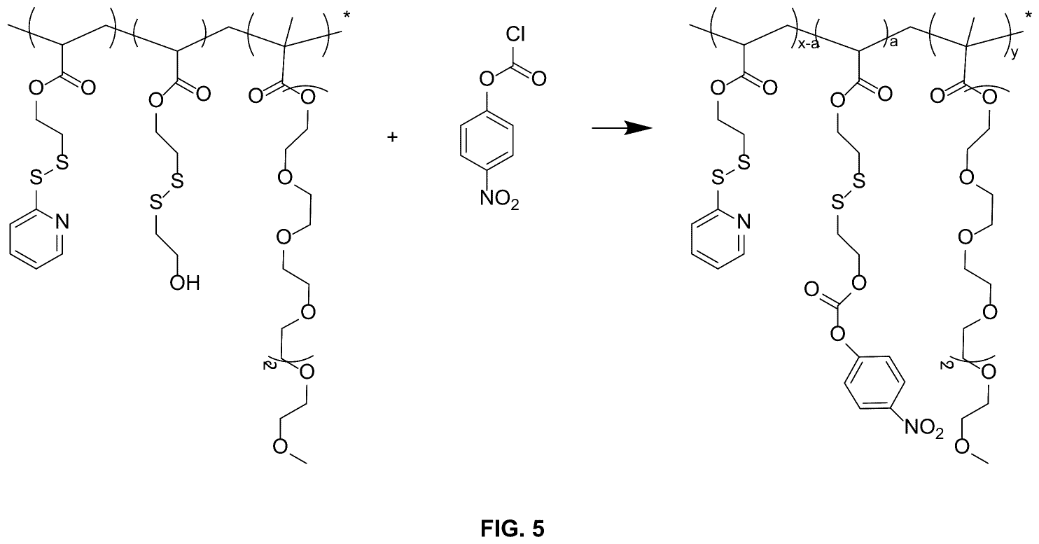

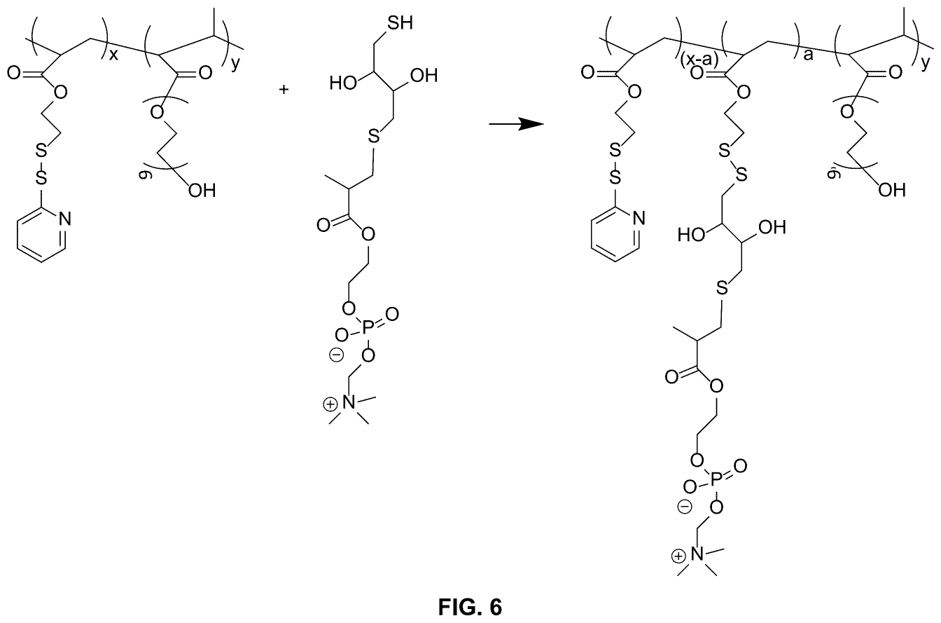

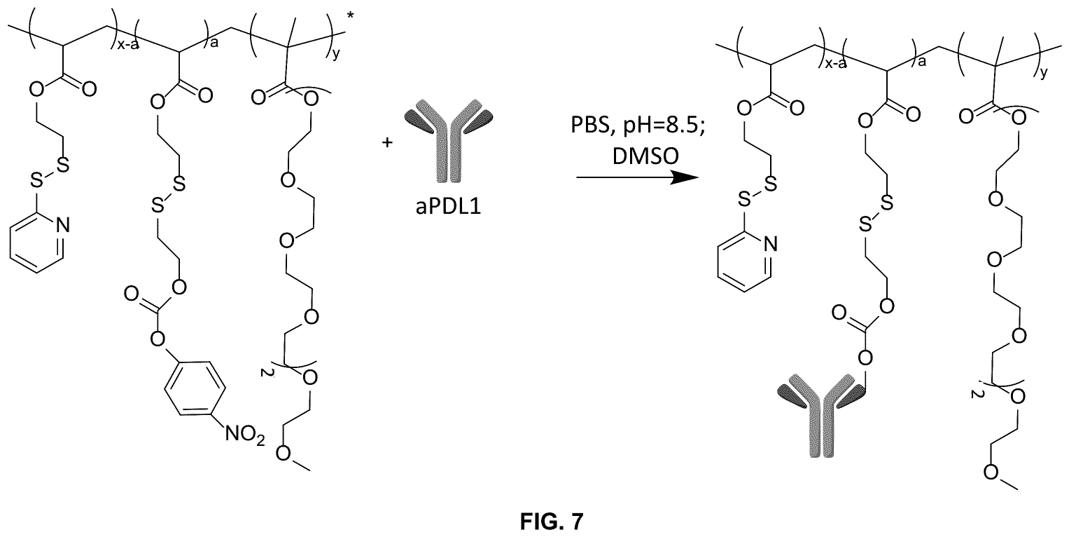

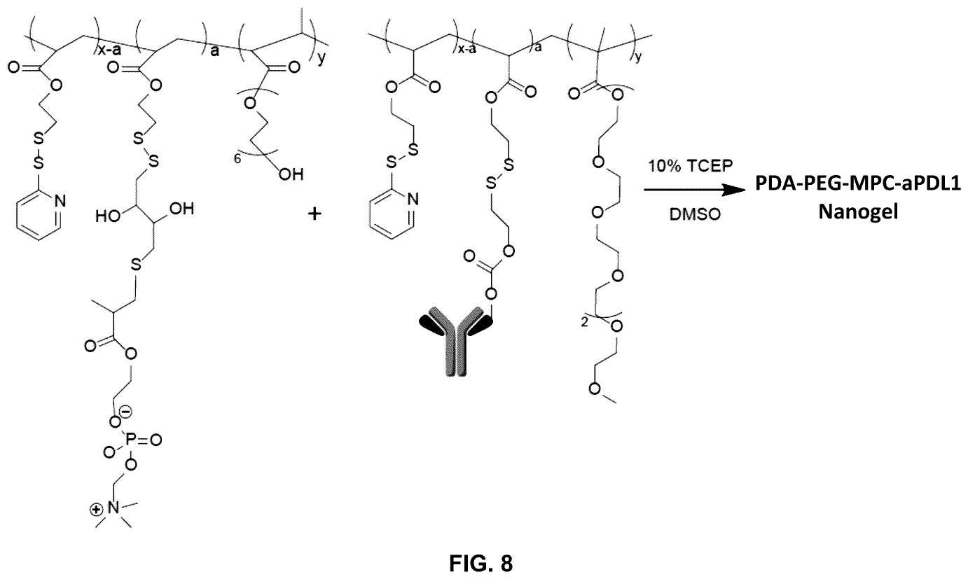

In one embodiment as illustrated in FIG. 4, a first intermediate copolymer formed by reaction of a poly(ethylene glycol) with a pyridine-2-thiol-containing monomer can be modified by reaction of a portion of the pyridine-terminated groups to form a second intermediate copolymer that includes hydroxyl-terminated groups. In further reaction of this second intermediate copolymer (e.g., as shown in FIG. 5), pendant hydroxyl-terminated groups can be reacted with the Y functionality of an activator (e.g., the halogen group of the activator in FIG. 5), with the activated pendant group of the resulting precursor copolymer being capable of conjugation with an antibody via the remaining X functionality (e.g., the nitrophenyl group of the activator in FIG. 5). In this embodiment, the poly(ethylene glycol) component of the first and second intermediate copolymers will generally not terminate in a hydroxyl group, so as to avoid activation of the poly(ethylene glycol) component by reaction with the activator group and to ensure that the activated pendant group is developed from a pyridine-terminated group of the initially formed copolymer.

Examples of materials that can react with a pyridine group to form a hydroxyl-terminated pendant group can include a mercapto alcohol, e.g., 2-mercaptoethanol (BME), 3-mercapto-1-butanol, etc., or other suitable reagents that can react via a thiol-disulfide exchange reaction to provide a terminal hydroxyl group (see, e.g., FIG. 3).

The terminal hydroxyl groups of the intermediate copolymer thus formed can be available for reaction with an activator to provide a precursor copolymer that includes a pendant leaving group, X, suitable for conjugation with an antibody. By way of example, X and Y may include, without limitation, a halide group, a mesyl group, a tosyl group, an aroxyl group such as a phenoxyl group, and a substituted aroxyl group such as a substituted phenoxyl group. Examples of activators include, but are not limited to, chloroformates, e.g., 4-nitrophenyl chloroformate, pentafluorophenyl chloroformate, succinimidyl chloroformate, or combinations thereof.

Following activation, a precursor copolymer can be conjugated to aPDL1 through reaction of the activated pendant group with the antibody under conditions as are known to those in the art (e.g., pH 8.5, etc.).

In addition to aPDL1 conjugated thereto, a nanogel can include a ligand for targeting nAchR. In one particular embodiment, this targeting ligand can include a phosphorylcholine group, which can function as a ligand for any neuronal subtype nAchR present on the BBB itself or on either side of the BBB, including both homomeric and heteromeric combinations of the twelve different nicotinic receptor subunits: α2-α10 and β2-β4. Examples of nAchR neuronal subtypes that can be targeted by a phosphorylcholine ligand of a nanogel can include, without limitation, (α₄)₃ (β2)₂, (α₄)₂ (β₂)₃, (α₃)2 (β₄)₃, α₄α₆β₃(β₂)₂, and (α₇)₅, as well as combinations thereof. In one particular embodiment, a phosphorylcholine ligand can target an α₇ nAchR that includes only α₇ subunits.

In one embodiment, a phosphorylcholine ligand can be incorporated to target nAchR as are expressed by microglial cells or astrocytes in therapeutic delivery of aPDL1 to the interior of the cells. A phosphorylcholine group can also facilitate crossing of the BBB in conjunction with a RAGE targeting ligand.

In formation of a copolymer, a precursor copolymer (e.g., a PDA-PEG copolymer as initially formed) can be reacted with a monomer that includes a phosphorylcholine group. Alternatively, a PDA-PEG copolymer can be an intermediate copolymer that can be further modified to form a precursor copolymer that includes suitable functionality for reaction with a monomer that includes a phosphorylcholine groups.

In one embodiment, a phosphorylcholine group-containing monomer can include the general structure of:

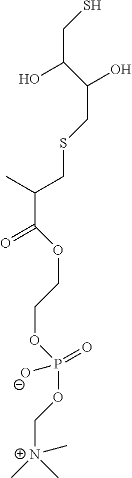 [SMILES: CC(CSCC(O)C(O)CS)C(=O)OCCOP(=O)([O-])OC[N+](C)(C)C]

in which the phosphorylcholine head of the monomer includes only a single carbon between the ammonium group and the phosphate group, rather than a two-carbon ethyl group as is more common in phosphoryl choline monomers.

Of course, a phosphorylcholine-containing monomer is not limited to such an embodiment. Examples of a monomer including a phosphorylcholine group can include, without limitation, 2-acryloyloxyethyl phosphorylcholine, 2-methacryloyloxyethyl phosphorylcholine (MPC), 2-(meth)acryloyloxyethoxyethyl phosphorylcholine, 6-(meth)acryloyloxyhexyl phosphorylcholine, 10-(meth)acryloyloxyethoxynonyl phosphorylcholine, allyl phosphorylcholine, butenyl phosphorylcholine, hexenyl phosphorylcholine, octenyl phosphorylcholine, decenyl phosphorylcholine, or combinations thereof.

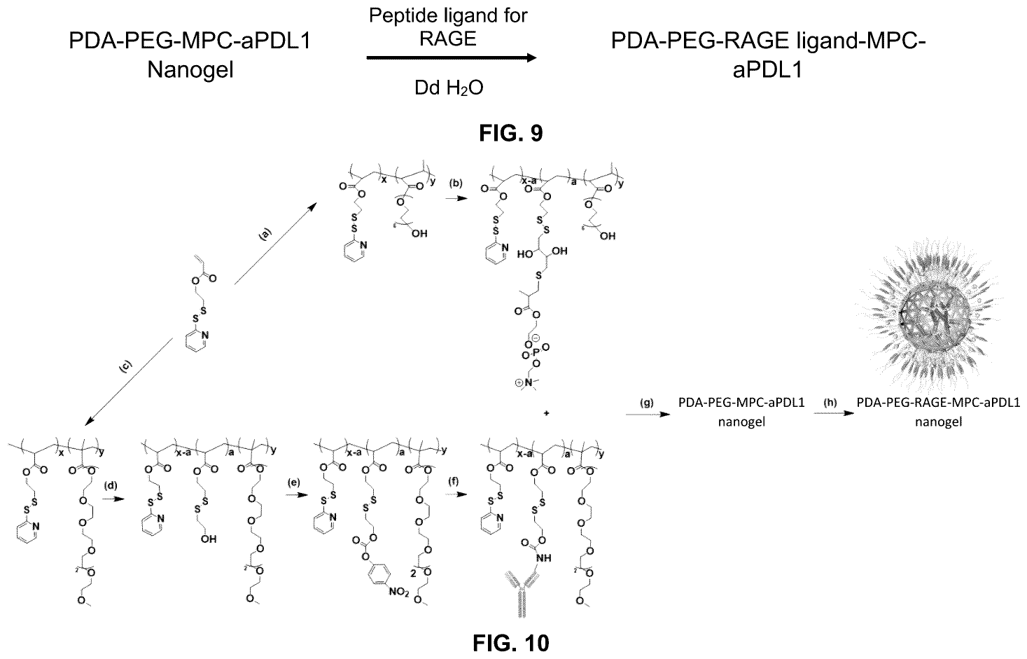

In the illustrated embodiment of FIG. 1 and FIG. 9, the RAGE targeting ligand can be conjugated to the external surface of the crosslinked nanogel. Rage targeting peptides or small molecules are encompassed herein include those targeting one or more domains (e.g., V, C1, or C2 domains), receptor isoforms, or polymorphisms. By way of example, and without limitation, a RAGE targeting ligand can include a relatively low molecular weight advanced glycation endproduct such as Na-carboxy-methyl-lysine (CML), NE-carboxy-ethyl-lysine (CEL), quinolinic acid, and methylglyoxal-derived hydroimidazolones (MG-H) as well as larger peptide-based structures including Aβ (e.g., Aβ₁₋₄₀ and Aβ₁₋₄₂), HMGB1, S100/calgranulins, Aβ fibrils, and cross-linked/modified Aβ.

The nanogels can be administered to a subject in need thereof as a component of a pharmaceutical composition in a pharmaceutically effective amount.

The term “pharmaceutically effective amount” refers to the amount of a drug or pharmaceutical agent that will elicit the biological or medical response of a tissue, system, animal, or human that is being sought by a researcher or clinician. This amount can be a therapeutically effective amount.

The term “pharmaceutically acceptable carrier” is used herein to refer to a carrier that is useful in preparing a pharmaceutical composition that is generally safe, non-toxic and neither biologically nor otherwise undesirable, and is acceptable for veterinary use as well as human pharmaceutical use. A “pharmaceutically acceptable carrier” as used in the specification and claims can include both one and more than one such carrier. The term “pharmaceutically acceptable” refers to a carrier that is compatible with the other ingredients of the formulation and not deleterious to the recipient thereof.

The terms “administration of” or “administering a” pharmaceutical composition should be understood to mean providing a pharmaceutical composition to an individual in need of treatment in a form that can be introduced into that individual's body in a therapeutically useful form and therapeutically useful amount, including, but not limited to: oral dosage forms, such as tablets, capsules, syrups, suspensions, and the like; and injectable dosage forms, such as IV, IM, or IP, and the like.

Pharmaceutical compositions may be prepared by any of the methods well known in the art of pharmacy. Pharmaceutical compositions encompass any compositions made by admixing the nanogels and a pharmaceutically acceptable carrier, optionally in conjunction with one or more additional components as are generally known in the art. The carrier may take a wide variety of forms depending on the form of preparation desired for administration, e.g., oral or parenteral (including intravenous). Thus, the pharmaceutical composition can be presented as discrete units suitable for oral administration such as capsules, cachets, or tablets each containing a predetermined amount of the active ingredients. Further, the composition can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the composition may also be administered by controlled release means and/or delivery devices. The foregoing list is illustrative only and is not intended to be limiting in any way.

Pharmaceutical compositions intended for oral use may contain one or more agents selected from the group consisting of sweetening agents, flavoring agents, coloring agents and preserving agents in order to provide pharmaceutically elegant and palatable preparations. Tablets may contain a composition having at least one of the compounds described herein in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. A tablet may be prepared by compression or molding, optionally with one or more accessory ingredients or adjuvants. Compressed tablets may be prepared by compressing, in a suitable machine, at least one of disclosed compounds in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions for oral use may also be presented as hard gelatin capsules wherein one or more of the disclosed compounds is mixed with an inert solid diluent, for example, calcium carbonate, calcium phosphate or kaolin, or as soft gelatin capsules wherein the compound(s) is/are mixed with water or an oil medium, for example peanut oil, liquid paraffin, or olive oil.

Pharmaceutical compositions can also include aqueous suspensions, which contain the nanogels in admixture with excipients suitable for the manufacture of aqueous suspensions. In addition, oily suspensions may be formulated by suspending the nanogels in a vegetable oil, for example arachis oil, olive oil, sesame oil or coconut oil, or in a mineral oil such as liquid paraffin. Oily suspensions may also contain various excipients. The pharmaceutical composition may also be in the form of oil-in-water emulsions, which may also contain excipients such as sweetening and flavoring agents.

Pharmaceutical compositions can be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy injection. The pharmaceutical compositions must be stable under the conditions of manufacture and storage, and should be preserved against the contaminating action of microorganisms such as bacteria and fungi.

Beneficially, disclosed brain-targeted aPDL1-loaded nanogels can effectively cross the BBB, deliver aPDL1 into microglia and astrocytes, release the aPDL1 intracellularly, subsequently deplete PD-L1 in the microglia and astrocytes, and revive cell function in clearing Aβ and attenuating neuroinflammation in AD brain. Consequently, disclosed materials can be beneficially utilized in stopping the progression of AD and retain the learning and cognitive capacity.

Disclosed materials and methods offer a promising strategy for modulating the brain's immune response and addressing the underlying pathology of neurodegenerative disorders.

The present invention may be better understood with reference to the Example, set forth below.

### Example

### Chemicals

Cyanine 5 NHS ester (Cy5-NHS) was purchased from Lumiprobe Co. (Cockeysville, MD, USA). 6.5 mm Transwell® with 0.4 μm Pore Polycarbonate Membrane Insert (Costar, 3413) was purchased from Fisher Scientific. Amyloid-beta 42 peptide was purchased from GL Biochem (Shanghai, Catalog No. 052487). 1,1,1,3,3,3-hexafluoroisopropanol (HFIP) and (3-(4,5-dimethylthia-zol-2-yl)-2,5-diphenyltetrazolium bromide (MTT) was purchased from Tokyo Chemical Industry Co., Ltd (Portland, OR, USA). Fluorescein isothiocyanate (FITC) and Thioflavin S (ThS) were acquired from Sigma-Aldrich Chemical Co. (St. Louis, MO, USA).

### Cell Culture

BV2 (ATCC; HTB-26), were cultured in Dulbecco's modified Eagle's medium (DMEM) (Thermo Fisher Scientific), supplemented with 10% v/v Fetal bovine serum (FBS) (Cytiva, Cat no. SH30396.03) and 1% v/v Penicillin-Streptomycin with 10,000 units penicillin (PS) (P0781, Sigma). Primary human astrocytes (Sciencell, Cat no. 1800) and primary mouse astrocytes (Sciencell, Cat no. 1800-57) were maintained at 1% v/v FBS and 1% v/v Penicillin-Streptomycin with 10,000 units penicillin (PS) supplemented astrocyte medium (Sciencell, Cat no. 1801), astrocyte growth supplement (Sciencell, Cat no. 1852). All the cells were incubated at 37° C. in a 5% CO₂ incubator.

### Nanogel Fabrication and Characterization

PDA-PEG-IgG (Poly-IgG) and PDA-PEG-PDL1 (Poly-PDL1) were synthesized as described above and as illustrated in FIG. 10. In general, the scheme included: (a) reaction of poly(ethylene glycol) methacrylate (PEG360), 2′2-azobisisobutyronitrile (AIBN), anisole, 65° C. (b) further reaction with MPC-SH, r.t. (c) reaction of poly(ethylene glycol) methyl ether methacrylate (PEG500), 2′2-azobisisobutyronitrile (AIBN), anisole, 65° C. (d) further reaction with 2-Mercaptoethanol (BME), dichloromethane, r.t. (e) followed by reaction with 4-Nitrophenyl Chloroformate, pyridine, dichloromethane, 4° C. shift to r.t. (f) followed by conjugation with aPDL1, PBS buffer (pH=8.4), dimethyl sulfoxide (DMSO), 4° C. (g) crosslinking of the functionalized copolymers in the presence of Tris(2-carboxyethyl) phosphine Hydrochloride (TCEP), dimethyl sulfoxide (DMSO), r.t. (h) surface functionalization of the crosslinked nanogels with RAGE peptide-Mal, dd H₂O, 4° C.

At step (g), the PDA-PEG-MPC was mixed with Poly-antibody followed by the addition of 5% (tris(2-carboxyethyl) phosphine (TCEP) under stirring for cross-linking in DMSO at room temperature for 15 min. Next, the reaction mixture was added dropwise into dd H₂O on ice under stirring to form the nanogel. The nanogel solution was further dialyzed against dd H₂O at 4° C. for 3 times. Then, the solution was concentrated by Spectra/Por® dialysis tube (regenerated cellulose, MWCO: 100 kDa). RAGE targeting peptide (w/w 2% of polymer dissolved in DMSO) was added to the nanogel solution and string at 4° C. for 24 h. Next, the nanogel solution was used for dialysis with dd H₂O at least 3 times to remove the free peptide and DMSO with MW 8000 kDa dialysis bag at 4° C. Finally, the solution was concentrated again by Spectra/Por® dialysis tube and storage at 4° C.

Nanogel suspension was loaded onto a 300-mesh copper grid and air-dried. The morphology of the nanogel particles was captured with a transmission electron microscope (Hitachi HT7800 TEM, Hitachi High Technologies, Tokyo, Japan). The hydrodynamic size and zeta potential of the nanogels were measured by a Zetasizer (Nano-ZS).

### Polyacrylamide Gel Electrophoresis of PDDA-PEG-aPDL1

To confirm that the antibodies were successfully conjugated onto the polymer polyacrylamide gel electrophoresis was adopted. To mimic the intracellular microenvironment, 2 mM GSH was added to release the antibodies from the polymer-antibody conjugates. After 90 min of electrophoresis, Coomassie brilliant blue was used to stain the gel for 1 h and washed with water until bands were clear, at r.t. The migration of the bands on the gel was imaged by a ChemiDoc™ system (BIO-RAD).

### Cytotoxicity Assay

The MTT assay was used to determine the effect of the nanogels on protecting cells from damage from LPS. BV2 cells were seeded in 96-well plates (8,000 cells/well) at 37° C., 5% CO₂, and cultured overnight to allow the attachment of the cells. Cells were incubated with a complete medium supplemented with 100 ng/mL LPS for 3 hrs. After that, aPDL1 or nanogels were added and incubated for another 48 hrs. The viability of the cells after treatments were evaluated with MTT assay by measuring the absorbance at 570 nm with Molecular Devices SpectraMax i3x Microplate Reader. Data were analyzed and plotted by GraphPad Prism 9.4.1.

### Aβ Oligomer-FITC Labeling and the Formation of Aβ Aggregates

Lyophilized Aβ₄₂ peptide was first dissolved to a final concentration of 5 mg/mL in 1,1,1,3,3,3-hexafluoroisopropanol (HFIP). After that, the solution was sonicated for 20 minutes before being shaken for 2 hours at room temperature. The HFIP was removed under a gentle nitrogen flow and stored at −20° C. Aβ₄₂ peptide samples were reconstituted fresh before each use to avoid self-assembly. After dissolving the sample in DMSO, it was sonicated at room temperature for 10 min before being diluted in PBS for use as the stock solution. For the FITC labeling, the AB oligomer (1 mg) and FITC (0.1 mg) were reacted in DMSO and stirred at room temperature. After 24 h, the solution was dialysis against DMSO (dialysis bag MW 1,000 kDa) 3 times to remove free FITC. During this time, the Aβ oligomer should assemble to form Aβ aggregates.

### BV2 Cell Uptake of Aβ-FITC

BV2 cells were seeded in a glass bottom round dish and cultured overnight. The above prepared Aβ-FITC aggregates were added to the cells and incubated for 3 hrs. After that, different treatments were added to the cells without removing the old media. Images of the cells were taken at different time points for 3, 8, and 24 hrs with the confocal microscope.

### Flow Cytometry

HCMEC/d3, BV2, and MA cells were cultured in 6-well plates. After adding blank, aPDL1-Cy5, and BTN-PDL1-Cy5 to the cells, they were incubated at 37° C. in a cell culture incubator for 3 hours. The cells were harvested using 0.25% trypsin for 3 minutes and fixed with 4% paraformaldehyde (PFA) at room temperature for 20 minutes. The cells were washed three times with PBS, resuspended in 200 μl of PBS, and analyzed using the BD LSRFortessa flow cytometer (BD Biosciences). Flow cytometry data analysis was performed using FACS Diva software (Becton, Dickinson and Company).

### Transwell Endothelial Cells HCMEC/d3 Mimicking BBB Model Penetration of the BTN-IgG-Cy3

Transwell inserts (0.4 cm²) were placed in a 24-well companion plate and incubated at 37° C. for 1 hour using collagen. The inserts were then washed once with PBS. Next, 1×10⁵ HCMEC/d3 cells were seeded in 200 μL of the complete endothelial medium in each insert, while 500 μL of the complete endothelial medium was added to the lower layer. Trans-endothelial electrical resistance (TEER) measurements were performed daily until TEER readings stabilized, indicating the establishment of the BBB integrity. For the treatment groups, including blank, BSA-Cy3, and BTN-PDL1-BSA-Cy3, samples were treated with complete medium, complete medium supplemented with BV2 cell supernatant medium concentrate, or complete medium supplemented with LPS-activated BV2 cell supernatant medium concentrate. At different time points, samples were collected, and the fluorescence intensity was measured at an excitation wavelength of 550 nm and an emission wavelength of 562 nm to calculate the penetration efficiency.

### Immunoblotting

The BV2 cells were cultured in 6 well plates. LPS was pretreated for 3 hours for the cells and followed with different treatments. The cells were collected, and the protein was extracted with an extraction reagent (Thermo Scientific, catalog no. 78501). The BCA quantification kit was determined, and bromophenol blue was added to adjust the final protein concentration at 1 mg/mL. The samples were boiled at 95° C. for 15 min, then returned to room temperature. Protein (20 μg/well) was loaded in the 10% PAGE-GEL and run the electrophoresis under contain volt. The proteins were transferred to polyvinylidene fluoride (PVDF) membranes, blocked with 5% BSA for 1 hour at r.t, and incubated with primary antibodies at 4° C. overnight, followed by 1 hour at r.t incubation with HRP-conjugated secondary antibody. The blots were exposed under the ChemiDoc™ imaging system (BIO-RAD).

### ELISA Assay

The ELISA test was followed by the protocol for the Mouse TNF alpha uncoated ELISA kit (Invitrogen, catalog no. 88-7324-88) and Mouse IL-1 beta Uncoated ELISA Kit (Invitrogen, catalog no. 88-7013-88). The BV2 cells were seeded in 6 well plates with 20,000 cells/well until the cells were attached. 100 μg/mL LPS 100 μL/well was pretreated for 3 hours before adding different treatments. After 48 hours, the culture media were collected individually and centrifuged under 3,000 r.p.m for 10 min to remove the flowing cells. The medium was quantitated for nitrate/nitrite production.

### Therapeutic Effect of Nanogels in an AD Mouse Model

To investigate the therapeutic effect of the nanogels for AD, 5XFAD mice (8 month old) were randomly divided into four groups (n=7) and i.v. injected with saline, aPDL1, BTN-IgG, and nanogels (4 mg/kg) once per week for two weeks. Age-matched C57BL/6 mice in the normal group and AD mice in the control group were administrated with saline.

Biodistribution of aPDL1-Cy5 and Nanogel-Cy5

The test was performed on 8-month-old 5×FAD mice. The mice's fur was removed before they were injected with nanogel-Cy5 nanoparticles through the tail vein via i.v. The images were taken under the IVIS system at different time points. The biodistributions of the aPDL1 and BTN-PDL1 were determined in vivo by the IVIS Spectrum system. Four hours post injection, mice were sacrificed and perfumed with PBS and 4% paraformaldehyde. The brain, heart, liver, spleen, lungs, and kidneys were collected and imaged ex vivo with IVIS Spectrum system.

### Therapeutic Effect of Nanogels in an AD Mouse Model

To investigate the therapeutic effect of the nanogels for AD, 5XFAD mice (8-month-old) were randomly divided into four groups (n=7) and i.v. injected with saline, aPDL1, nanogels functionalized with IgG rather than PDL1, and nanogels as described herein (4 mg/kg) once per week for two weeks. Age-matched C57BL/6 mice in the normal group and AD mice in the control group were administrated with saline.

### Nesting Assay

The assessment of nesting capacity was performed once per week. Mice were individually housed in cages containing one square of pressed cotton at noon to test their nesting building behavior. The nesting results were photographed and scored the following morning. A 5-point nest rating scale, ex., 1, no nesting; 2, incomplete nesting; 3, flat nesting; 4, cup nesting; 5, dome nesting was adopted to characterize the constructed nests.

### Morris Water Maze Assay

This test was performed on 8-month 5×FAD mice. The swimming pool was made of blue polyethylene. On the southeast side of the pool, a clear plastic escape platform was located. The pool was filled to 10 cm above the platform, and the water was made opaque by milk. The water temperature was kept between 22 and 24° C. To eliminate competing environmental clues, lights were turned off the in the pool room, and four-floor lamps were set on the diagonal. A camera mounted on the ceiling in the center of the test chamber recorded the mice's behavior. The cameras were linked to an Etho Vision 3.1 computerized video tracking system. Before the final test, mice required 5 days of non-spatial training to acclimate to the pool and the underwater platform. Mice were dropped into the pool in four diagonal orientations for four 60-second swimming sessions separated by more than 30 minutes. After removing the platform on the sixth day, a random location was chosen, and the mice were placed individually in the swimming pool for testing. Data were collected and analyzed by ANOVA.

### Immunocytochemistry

Cells were treated with 4% paraformaldehyde for 30 min, followed by incubating with 0.1% Triton-X 100 (TX-100) for 3 min and blocking with 5% BSA buffer for 1 h at room temperature. After that, the samples were treated with primary antibodies at 4° C. overnight. The next day, the PBS-T was used to wash the samples 3 times/5 min. The samples were incubated with a fluorescence labelled secondary antibody for 1 h at room temperature and washed with PBS-T for 3 times. Then, the Hoechst 33342 was used to label the nuclei of the samples. Finally, the slides were covered mounting with Prolong™ Gold antifade reagent (Invitrogen, catalog, P36934) and sealed with cover glasses before being imaged with an EVOS™ FL microscope.

### Immunohistochemistry

Tissues were treated with 4% paraformaldehyde for 24 hours at r.t., followed by stepwise dehydration in 15% sucrose PBS solution and 30% sucrose PBS solution for other 24 h. The dehydrated tissues were embedded in the optimal cutting temperature (O.C.T, Fisher Healthcare, catalog, 4587) and frozen at −80° C. overnight. All the organs were cut in 12 μm slices with a cryo-microtome and stored at −20° C. The resulting tissue sections were incubated with 0.1% TX-100 for 3 min and blocked under 5% BSA buffer for 1 hour at room temperature. After that, the samples were treated with primary antibodies at 4° C. overnight. The next day, the PBS-T was used to wash the samples 3 times/5 min. The samples were incubated with a fluorescence labelled secondary antibody for 1 h at room temperature and washed with PBS-T for 3 times. Then, the Hoechst 33342 was used to label the nuclei of the samples. Finally, the slides were covered by mounting with Prolong™ Gold antifade reagent (Invitrogen, catalog, P36934) and sealed with cover glasses before imaged with an EVOS™ FL microscope.

### Hematoxylin and Eosin (H&E) Stain

The freshly sliced tissue sections were allowed to recover to room temperature, and the O.C.T was removed by washing with PBS 3 times/15 min. Cooled acetone was used to fix the tissues for 5 min. Then, the slices were stained with an H&E staining kit. After that, the slides were mounted with Cytoseal™ XYL (Epredia, catalog, 8312-4), and imaged with an Amscope microscope.

### Statistical Analysis

All data were displayed as mean±standard deviation (SD) (n≥3), and the statistical significance was analyzed by GraphPad Prism 9.0 (GraphPad Prism Software Inc., San Diego, California) using Student's t-test or ANOVA with Tukey's significant. Differences were considered significant when the p-value was less than 0.05.

### Results

### Characterization of the Nanogels

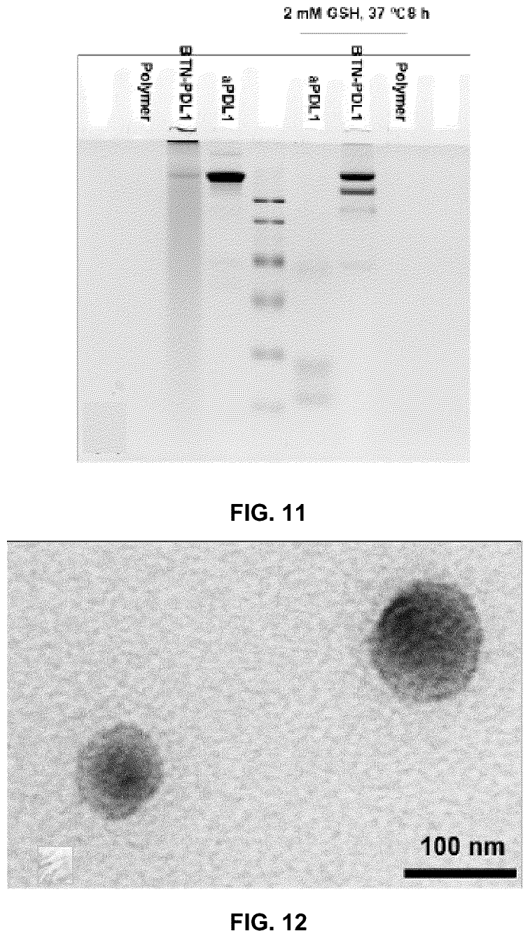

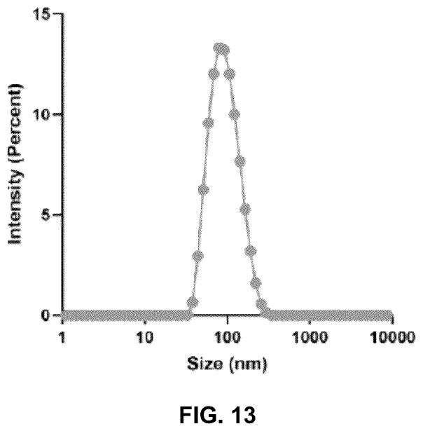

To confirm the successful encapsulation of aPDL1 into nanogels and its release under a reducing environment mimicking intracellular conditions, agarose gel electrophoresis was conducted. Results are shown in FIG. 11. Lane 4 of FIG. 11 was a protein marker. Samples in lanes 5-7 (aPDL1, BTN-PDL1, and polymer) were incubated in 1 mM GSH and 37° C. for 1 hour. The nanogel's size distribution was determined to be 98.43+41.43 nm (PDI: 0.16, as shown in FIG. 13), and its morphology was spherical, as determined by transmission electron microscopy (FIG. 12). The surface charge of BTN-PDL1 was determined to be −14+9.32 mV by zeta sizer.

### Cellular Uptake of TN-PDL1 by Brain Endothelia, Microglia, and Astrocytes

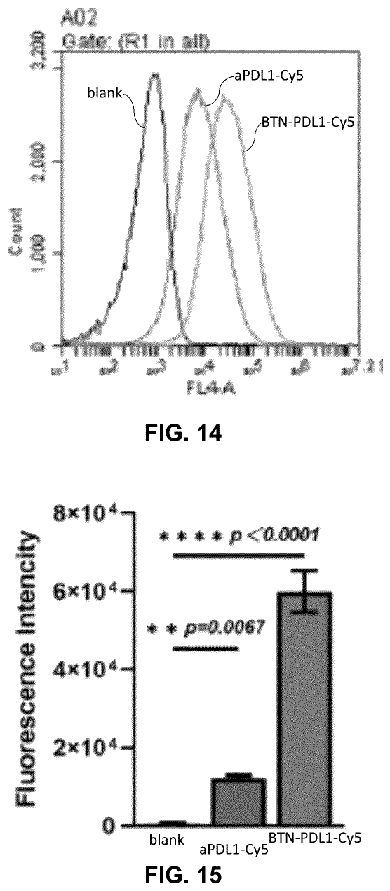

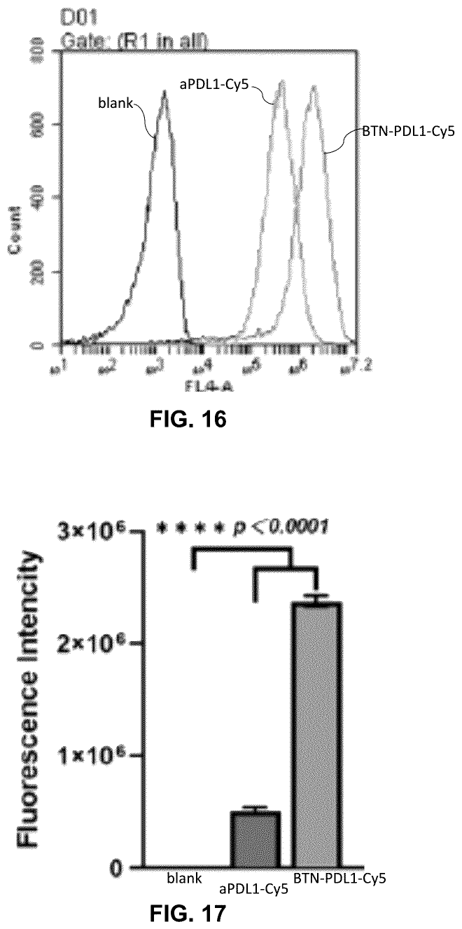

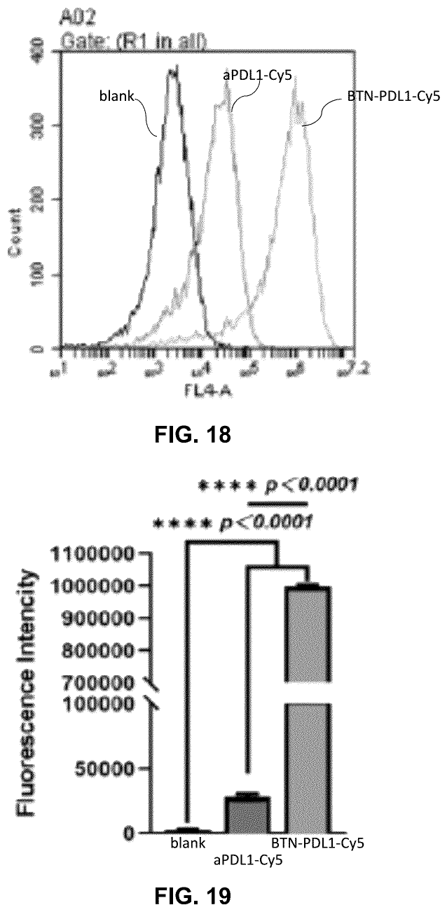

To probe the cellular uptake of the nanogels by HCMEC/d3, BV2, and mouse astrocytes (MA) cells, flow cytometry analysis was employed. FIG. 14 and FIG. 15 show results for HCMEC/d3 cells, FIG. 16 and FIG. 17 show results for BV2 cells, and FIG. 18 and FIG. 19 show results for MA cells. As illustrated, the results revealed that, compared with aPDL1, BTN-PDL1 exhibited significantly higher efficacy in entering hCMEC/D3, BV2, and MA cells. Standing alone red signals in confocal images further proved that BTN-PDL1 could effectively escape from the lysosomes after entering microglia, which may be due to a proton sponge effect induced by the protonated PDA groups under an acidic lysosomal environment.

### Assessing the Penetration Efficiency of BTN-BSA-Cy3 Nanoparticles

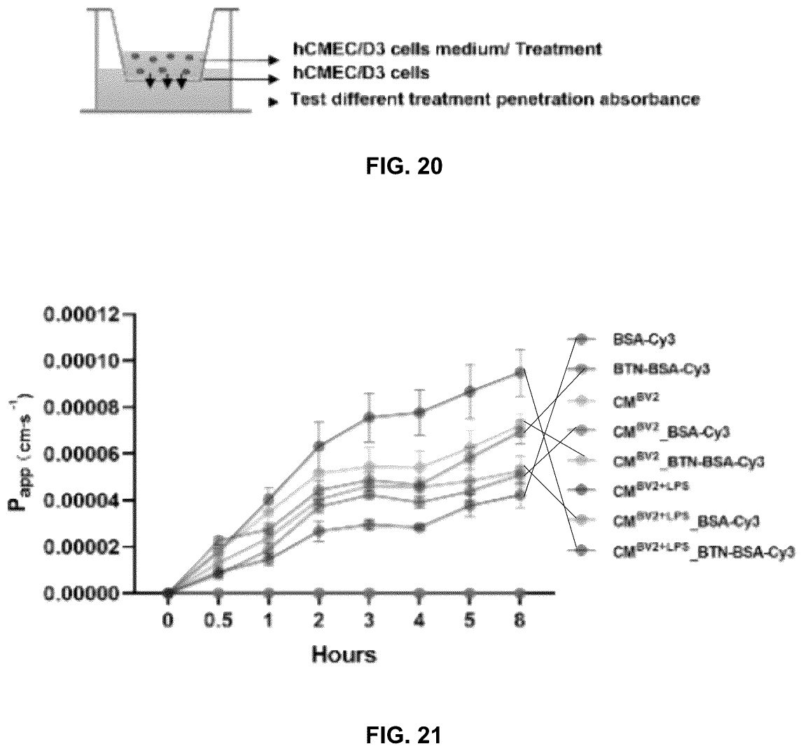

In order to investigate the ability of the nanogels in penetrating the BBB, the penetration efficiency of TN-BSA-Cy3 nanoparticles was assessed in an in vitro BBB Transwell model (FIG. 20). Nanoparticle delivery was evaluated in vitro using the transwell model in which brain microvascular endothelial cells (HCMEC/d3) were cultured on a porous filter membrane in the upper chamber. The integrity of the in vitro BBB was determined by measuring trans-endothelial electrical resistance (TEER), while the penetration capacity was assessed by detecting the fluorescence signals of Cy3-labeled nanoparticles. As indicated in FIG. 21, it was found that TN-BSA exhibited much higher BBB penetrating efficacy than free BSA. Interestingly, after the stimulation of conditioned medium from activated BV2 medium, which can induce the upregulation of the RAGE receptor on the BBB, improved penetration of TN-BSA was observed. In contrast, only a slight increase was noticed for the penetration of free BSA. Thus, as shown in FIG. 21, it was validated that the TN-based nanocarrier could effectively utilize the overexpressed RAGE to penetrate the BBB under an inflammatory environment.

### Assessment of RAGE Expression in Brain Blood Vessels of 5×FAD Mice

To confirm the overexpression of RAGE expression in the BBB of AD mice, immunofluorescent co-localization was conducted in brain sections of 8-month-old 5XFAD mice. The blood vessels and RAGE were detected with CD31 and RAGE antibodies, respectively. It was revealed that RAGE expression in the BBB was not noticeable in normal mice. In contrast, strong red signals overlapped with CD31 labeled blood vessels were observed in the brain slice of AD mice, suggesting that RAGE is a feasible target for nanoparticles effectively delivering aPDL1 into the brain.

### In Vivo Biodistribution Analysis of BTN-PDL1-Cy5

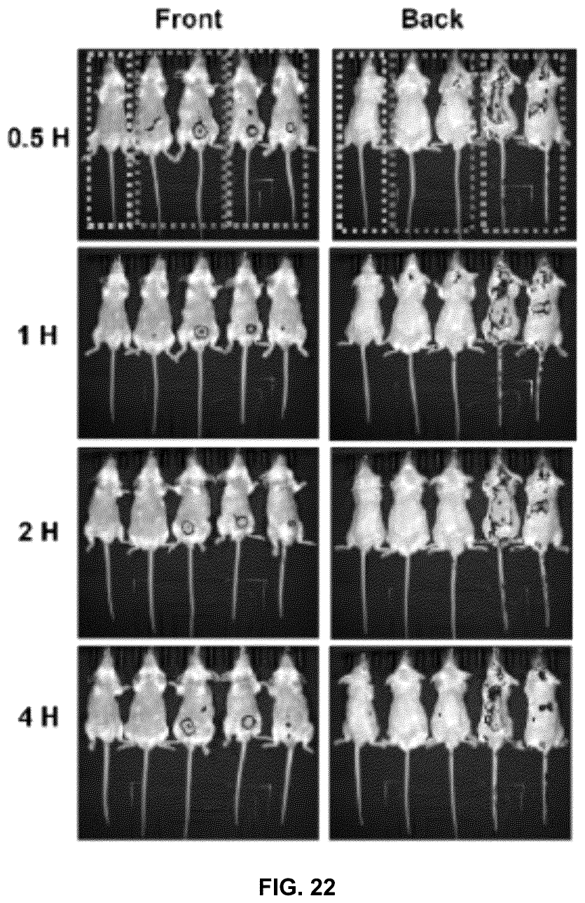

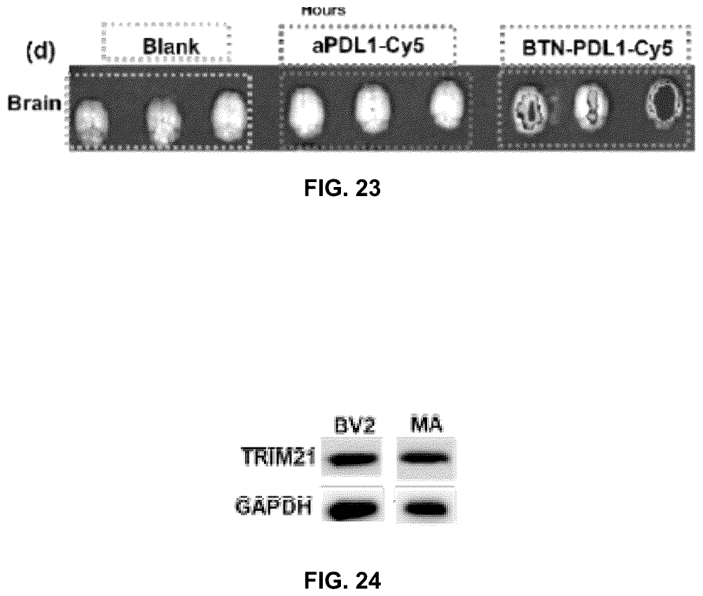

To further assess the biodistribution of aPDL1 in mice, fluorescent dye Cy5 was used to label aPDL1, and subsequently conjugated with PDA-PEG polymer before the fabrication of a nanogel. The biodistribution of this thus-formed BTN-PDL1-Cy5 was studied in 5×FAD mice using an IVIS® Spectrum In Vivo Imaging System (Perkin Elmer, Waltham, MA, USA). The excitation/emission wavelength pair of 610/650 nm provides the best signal-to-noise ratio and was therefore used for imaging of both the whole body and dissected organs. Whole-body images (FIG. 22) were acquired at 0.5, 1, 2, and 4 h post i.v injection (n=3 for each treatment). Afterwards, the major organs were harvested and ex vivo fluorescence signaling was acquired (FIG. 23, showing brain images). At 0.5 hours post injection, the fluorescence intensity of aPDL1-Cy5 mainly accumulated in the mouse bladder and was almost non-detectable in the brain, indicating its poor BBB penetrating capacity and rapid elimination. By contrast, strong fluorescence signals of BTN-PDL1-Cy5 were observed both in vivo and ex vivo in the brain of the animals, indicating the outstanding brain targeting effect of BTN-PDL1. To further probe whether the BTN-PDL1 could penetrate BBB, the collected brains were sectioned and stained with CD31 and DAPI. Compared with the faint red signals in brain slices from aPDL1-Cy5 treated mice, abundant red spots away from the green signal were observed in the brain slices from BTN-PDL1-Cy5 treated mice, validating its outstanding BBB penetrating capacity in an AD mouse model.

### TRIM21 Expression in Microglia and Astrocytes

Since the presence of TRIM21 is understood to be desirable for improved success of disclosed methods, TRIM21 expression was evaluated in both microglia and astrocytes. As shown in FIG. 24, it was confirmed that TRIM21 is highly expressed in both microglia and astrocytes.

### BTN-PDL1 Downregulates PD-L1 in Microglia

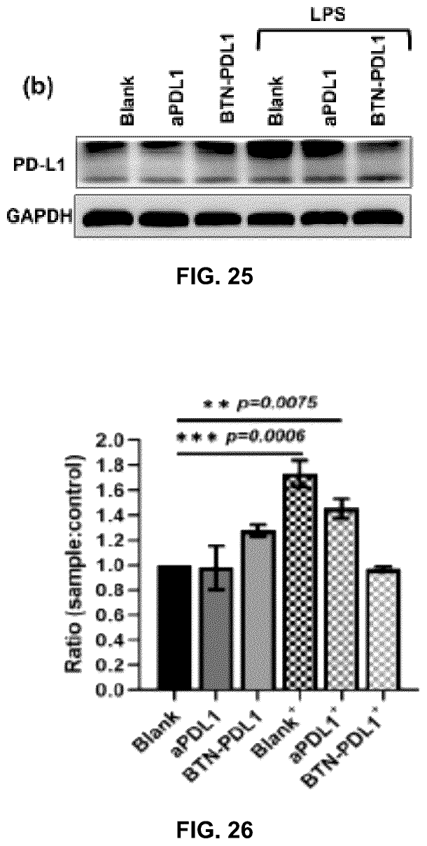

To investigate whether BTN-PDL1 exhibits a PD-L1-reducing effect in microglia, LPS was employed to trigger the activation of BV2 cells. As indicated in FIG. 25 and FIG. 26, it was revealed that LPS induced the upregulation of PD-L1 in microglia, validating the PD-L1 boosting effect of an inflammatory condition. Interestingly, as indicated in FIG. 25 and FIG. 26, 1 μg/ml of BTN-PDL1 led to the reduction of PD-L1 expression to normal levels in microglia under the stimulus of LPS, indicating the success of PD-L1 degradation via disclosed methods in microglia.

### BTN-PDL1 Downregulates PD-L1 in Astrocytes

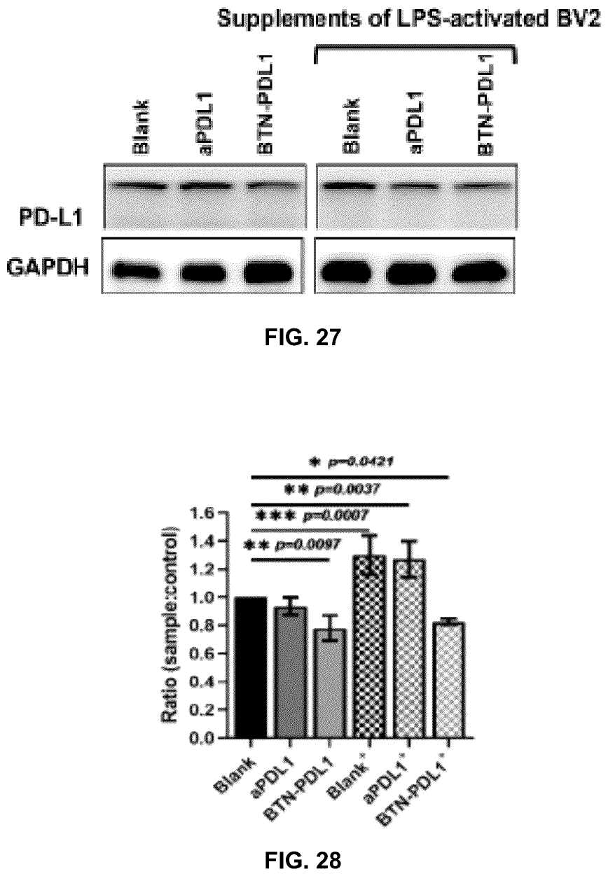

To investigate whether BTN-PDL1 exhibits a PD-L1-reducing effect in astrocytes, conditioned medium of LPS-stimulated BV2 cells (CM^{BV-2+LPS}) was utilized, known to contain cytokines that can activate astrocytes and upregulate PDL1 expression. As indicated in FIG. 27 and FIG. 28, it was revealed that CM^{BV-2+LPS} induced the upregulation of PD-L1 in astrocytes. Similar to its effect on microglia, 1 μg/ml of BTN-PDL1 also led to the reduction of PD-L1 expression to normal levels in astrocytes under the stimulus of CM^{BV-2+LPS}, indicating the success of PD-L1 degradation via BTN-PDL1 based Trim-Away in astrocytes.

### BTN-PDL1 Protects BBB Integrity

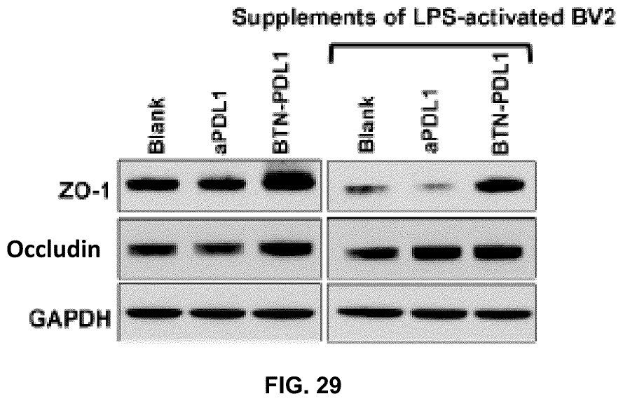

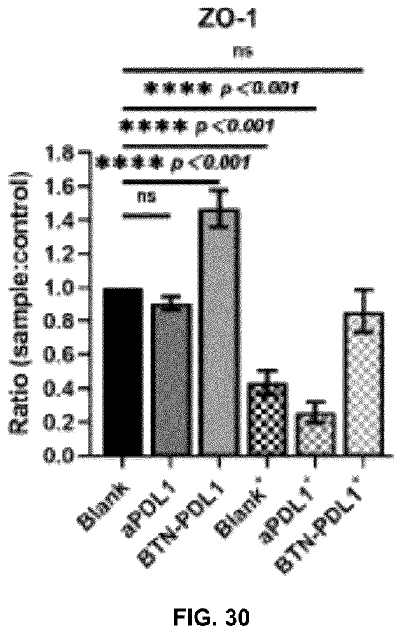

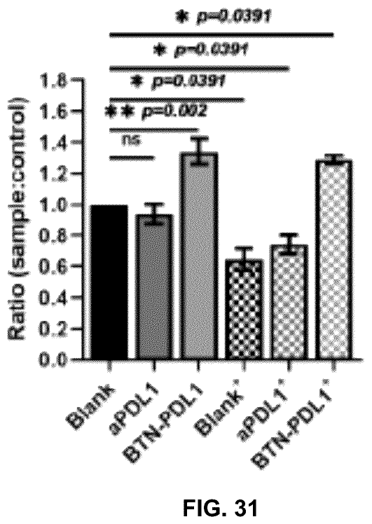

To further ascertain the safety of BTN-PDL1 without compromising the blood-brain barrier (BBB), the expression of endothelial cell tight junction proteins, ZO-1 and Occludin were examined. Results are shown in FIG. 29, FIG. 30, and FIG. 31. As indicated CM^{BV-2+LPS} induced the downregulation of ZO-1 and Occludin proteins, which is associated with the integrity of BBB. Surprisingly, BTN-PDL1 treatment restored ZO-1 and Occludin expression, suggesting the BBB protective effect of BTN-PDL1.

### BTN-PDL1 Inhibits Microglial Activation

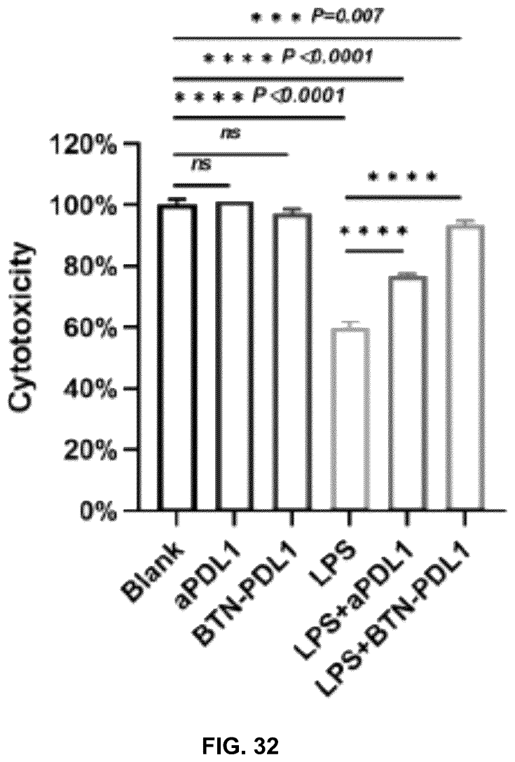

Immunofluorescence imaging indicated that BTN-PDL1 also downregulated the elevated PD-L1 induced by LPS stimulation. In addition, BTN-PDL1 attenuated the activation of microglia, evidenced by reduced IBA-1 expression. Furthermore, the viability of BV2 cells was significantly improved as indicated in FIG. 32.

### Neural Protective Effect of BTN-PDL1

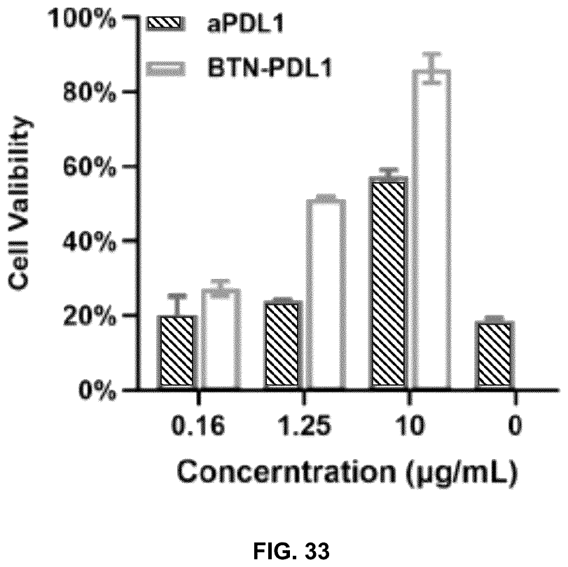

CM^{BV-2+LPS} induced the death of N2a differentiated neurons (FIG. 33), attributing to the releasing of toxic cytokines by activated microglia. It was surprisingly found that BTN-PDL1 exhibited an outstanding protective effect for the neurons treated with CM^{BV-2+LPS}.

### BTN-PDL1 Improves Microglial Capacity in Degrading Aβ

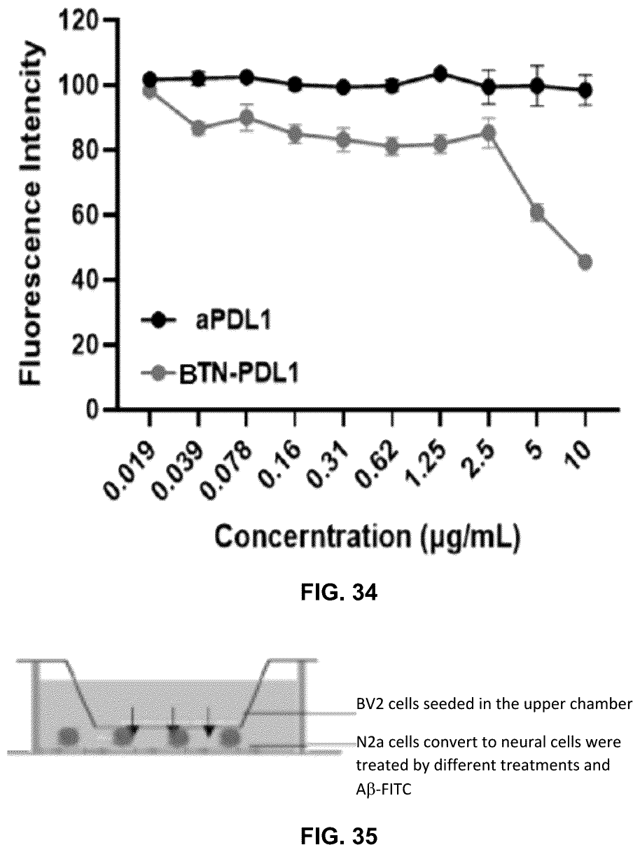

Further evaluation of the function of microglia after BTN-PDL1 indicated downregulation in microglia PD-L1. It was revealed that BTN-PDL1 treated microglia effectively engulfed Aβ within 3 h of incubation, evidenced by the colocalization of Aβ and lysosomes in fluorescence imaging. Furthermore, most of the engulfed Aβ signals disappeared in 24 h, suggesting the degradation of AB. In contrast, there was little or no change for Aβ incubated with aPDL1-treated BV2 cells (FIG. 34).

BTN-PDL1-Revived Microglia Protects Neurons from Aβ Damage

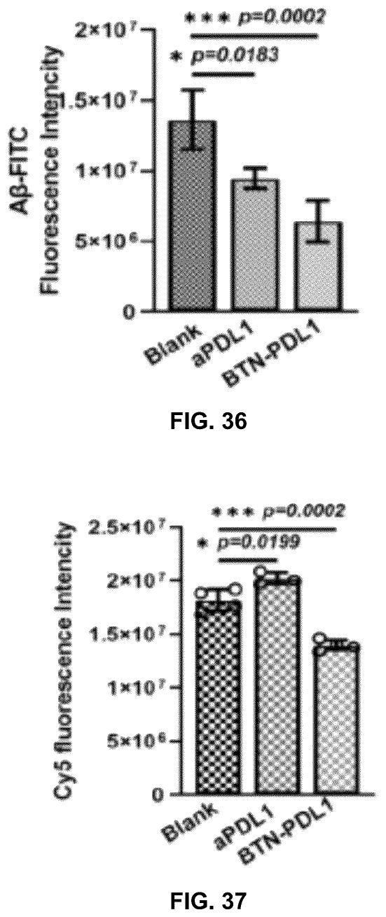

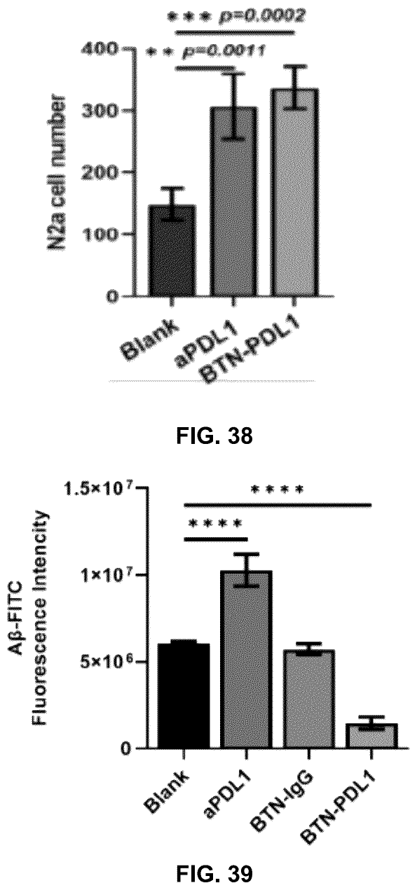

To determine if the BTN-PDL1-revived microglia could migrate and clear AB, a Transwell model (FIG. 35) was adopted. The appearance of Cy5 labeled BV2 and the reduced Aβ-FITC in the bottom chamber evidenced the activity of the revived microglia upon BTN-PDL1 treatment (FIG. 36, FIG. 37). It is also worth mentioning that both the number and the morphology of the neurons in the BTN-PDL1 treated group were significantly improved (FIG. 38).

### BTN-PDL1 Improves the Capacity of Astrocytes in Engulfing and Degrading Aβ and the Cell Debris of Microglia

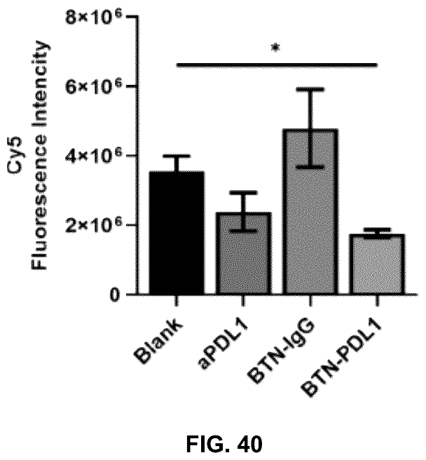

As is known, astrocytes can clear Aβ and the cell debris of microglia. The diminished BV2 debris and Aβ signals after 4 days of BTN-PDL1 treatment as indicated in FIG. 39 and FIG. 40 validated that revived astrocytes were able to engulf and degrade Aβ and microglial debris.

### Therapeutic Effect of BTN-PDL1 in an AD Mouse Model

### a) Nesting Assay

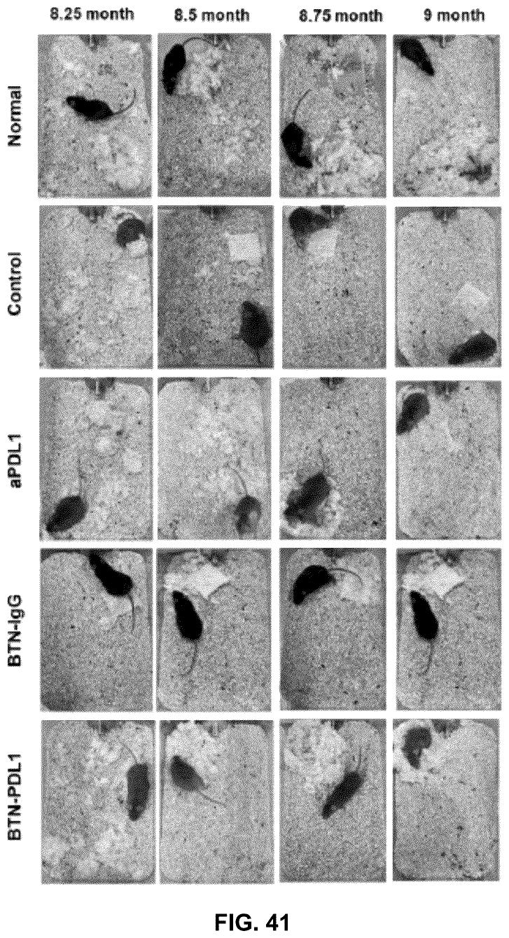

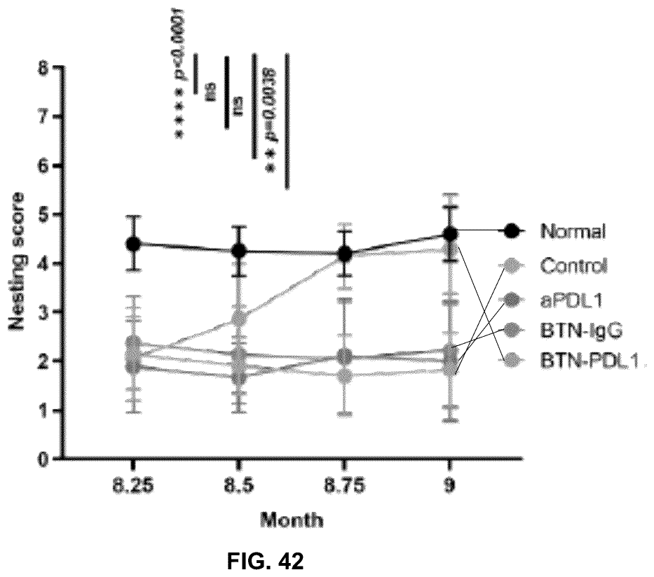

Nesting behavior is a widely used indicator of overall health. In this study, a previously established method for assessing nesting ability in mice was adopted. Specifically, 8-month-old 5×FAD mice were selected for the experiment. Each mouse was individually housed in a nest cage and provided with a 5×5 cm pressed cotton square overnight during the dark phase. Drug administration included a first i.v. injection at 8.25 months and a second injection at 8.5 months. Nest formation was evaluated before and 3 weeks after drug administration. As indicated in FIG. 41 and FIG. 42, the nesting skill for the mice in the control, aPDL1, and TN-IgG treatment groups remained unchanged. However, the BTN-PDL1 treated animals significantly improved their nesting skills.

### b) Morris Water Maze Assay

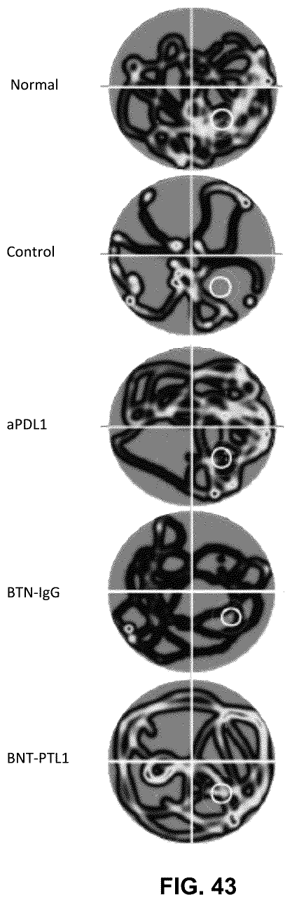

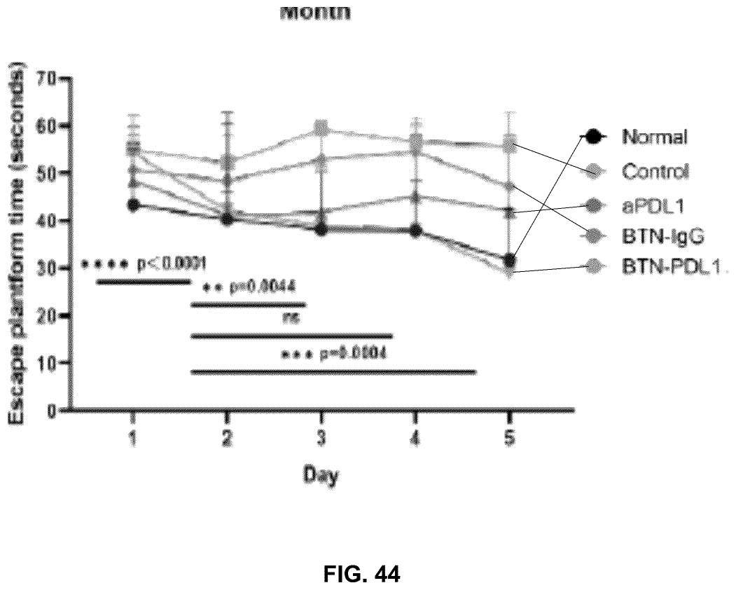

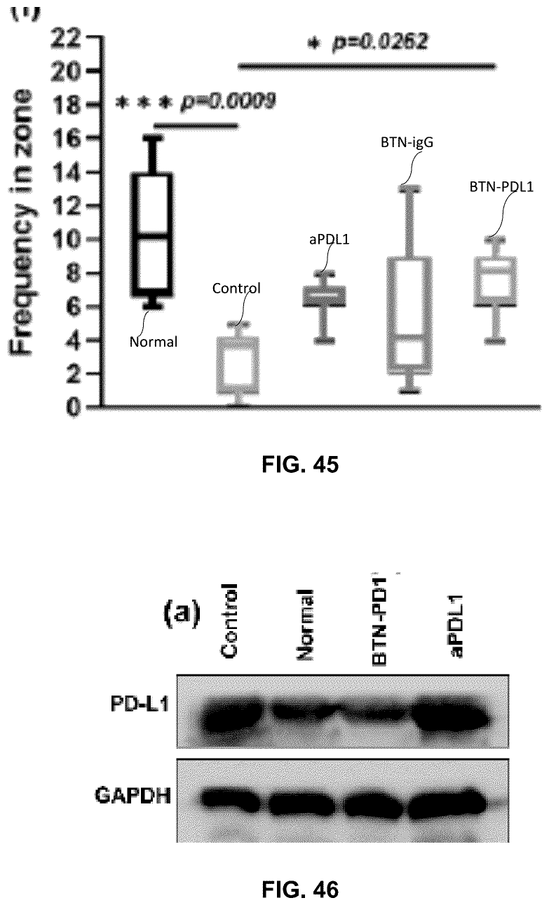

To evaluate the efficacy of BTN-PDL1 on the cognitive skill and memory in 5XFAD mice, Morris water maze (MWM) was employed. The performance of the mice was recorded through videos and analyzed by EthoVision® XT15 software to determine the latency to reach the platform and the number of targeted platform crossing for each mouse during the test. Interestingly, after two weeks of treatment, BTN-PDL1 treated AD mice spent most of the time in the targeted quadrant (FIG. 43), which is different from that shown in control, aPDL1, and BTN-IgG treated AD mice while similar to that shown in the normal mice. Furthermore, BTN-PDL1 treated AD mice showed similar learning capacity as the normal mice (FIG. 44), evidenced by the declined escape latency time during the training session. In addition, the increased number of crossing the targeted platform for the BTN-PDL1 treated AD mice suggested their improved memory ability as compared to that of the controls (FIG. 45).

### PD-L1 Expression in the Brain Tissue

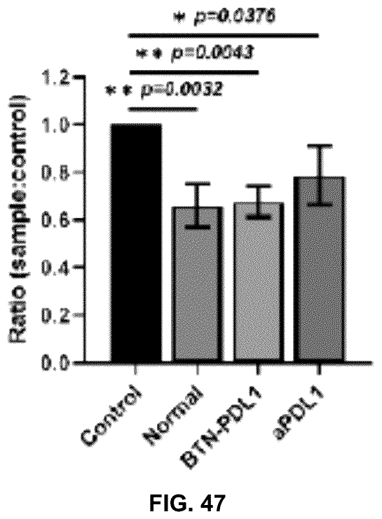

The complex relationship between programmed death ligand 1 (PD-L1) expression, neuroinflammation, and Aβ plaque clearance in the brain has garnered significant interest in recent research. In this study, the expression of PD-L1 in different groups of mice was investigated, shedding light on its potential role in those processes. It was revealed that there was a substantial increase in PD-L1 expression in the brains of the AD mice compared to that of age-matched normal ones. Intriguingly, a slightly attenuated increasing trend was observed in the aPDL1 treated AD group. However, BTN-PDL1 treatment effectively counteracted this effect by reducing PD-L1 expression and restoring it to a normal level (FIG. 46, FIG. 47).

To gain further insights into the distribution of PD-L1, brain slices from the mouse hippocampus were subjected to PD-L1/IBA1 immunobiological analyzed. This analysis revealed that microglia in the control group exhibited upregulated PD-L1, implying their involvement in the inflammatory response. This finding was supported by the colocalization of PD-L1 and activated microglia. Interestingly, the aPDL1 treated group did not display a reduction in PD-L1 expression, while the BTN-PDL1 treatment resulted in the disappearance of aggregated microglia along with a significant reduction of PD-L1 expression. A similar quenching effect on astrocyte activation was also noticed in the BTN-PDL1 treated mice, evidenced by a reduction of GFAP signals. At the same time, the diminished ThS stained Aβ plaques proved the revival of microglia and astrocytes after BTN-PDL1 treatment.

While certain embodiments of the disclosed subject matter have been described using specific terms, such description is for illustrative purposes only, and it is to be understood that changes and variations may be made without departing from the spirit or scope of the subject matter.

What is claimed is:

1. A method for reviving function in impaired microglia and/or astrocytes comprising delivering a nanogel to the microglia and/or astrocytes, the nanogel comprising a crosslinked network that includes a first copolymer and a second copolymer, the first copolymer comprising a first backbone, a first group pendant to the first backbone, the first group comprising a first poly(ethylene glycol), and a second group pendant to the first backbone that is conjugated to a PD-L1 antibody via a first disulfide bond, the second copolymer including a second backbone, a third group pendant to the second backbone, the third group comprising a second poly(ethylene glycol), and a fourth group pendant to the second backbone that is conjugated to a nictotinic acetylcholine receptor (nAChR) targeting ligand via a second disulfide bond, the nanogel further comprising a receptor for advanced glycation endproducts (RAGE) targeting ligand conjugated at a surface of the nanogel via a third disulfide bond; wherein
following delivery of the nanogel to the impaired microglia and/or astrocytes, the first, second, and third disulfide bonds are degraded, thereby releasing the PD-L1 antibody, and wherein
following release of the PD-L1 antibody, PD-L1 expressed by the microglia and/or astrocytes is degraded and the function of the impaired microglia and/or astrocytes is revived.

2. The method of claim 1, wherein the PD-L1 antibody is released within the microglia and/or astrocyte.

3. The method of claim 1, wherein the nanogel is delivered to the microglia and/or astrocytes in an environment that includes a pH of about 6.8 or less.

4. The method of claim 1, wherein the first copolymer comprises the reaction product of a third poly(ethylene glycol) with a first pyridine-2-thiol containing monomer and the second copolymer comprises the reaction product of a fourth poly(ethylene glycol) with a second pyridine-2-thiol containing monomer.

5. The method of claim 4, wherein the first pyridine-2-thiol-containing monomer and the second pyridine-2-thiol-containing monomer are independently selected from (pyridine-2-thiol)ethyl acrylate, (pyridine-2-thiol)ethyl methacrylate, N-(2-(pyridin-2-yldisulfanyl)ethyl) acrylamide, N-(2-(pyridin-2-yldisulfanyl)ethyl) methacrylamide, and ethyl(2-(pyridin-2-yldisulfanyl)ethyl) carbonate.

6. The method of claim 1, wherein the nAChR targeting ligand comprises a phosphorylcholine group.

7. The method of claim 6, wherein the fourth pendant group comprises the following structure:

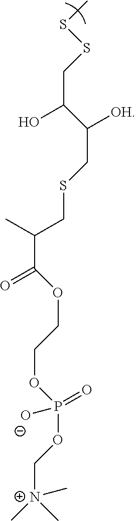 [SMILES: CSSCC(O)C(O)CSCC(C)C(=O)OCCOP(=O)([O-])OC[N+](C)(C)C]

8. The method of claim 1, wherein the RAGE targeting ligand comprises NE-carboxy-methyl-lysine, NE-carboxy-ethyl-lysine, quinolinic acid, a methylglyoxal-derived hydroimidazolone, an Aβ peptide, HMGB1, or an S100/calgranulins.

9. The method of claim 1, wherein the function of the microglia and/or astrocytes comprises degradation of Aβ.

10. The method of claim 1, wherein the function of the astrocytes comprises degradation of microglia cell debris.

11. The method of claim 1, wherein the function of the microglia and/or astrocytes comprises reduced activation of the microglia and/or astrocytes.

12. A method for treatment of a neurodegenerative disorder, the method comprising:
delivering a pharmaceutical composition to a subject, the composition comprising a nanogel and a pharmaceutically effective carrier, the nanogel comprising a crosslinked network that includes a first copolymer and a second copolymer, the first copolymer comprising a first backbone, a first group pendant to the first backbone, the first group comprising a first poly(ethylene glycol), and a second group pendant to the first backbone that is conjugated to a PD-L1 antibody via a first disulfide bond, the second copolymer including a second backbone, a third group pendant to the second backbone, the third group comprising a second poly(ethylene glycol), and a fourth group pendant to the second backbone that is conjugated to a nictotinic acetylcholine receptor (nAChR) targeting ligand via a second disulfide bond, the nanogel further comprising a receptor for advanced glycation endproducts (RAGE) targeting ligand conjugated at a surface of the nanogel via a third disulfide bond; wherein
following delivery of the pharmaceutical composition to the subject, the nanogel crosses the blood brain barrier; and wherein
the first, second, and third disulfide bonds are degraded following the crossing of the blood brain barrier, thereby releasing the PD-L1 antibody, and wherein
following release of the PD-L1 antibody, PD-L1 expressed by microglia and/or astrocytes is degraded and the function of impaired microglia and/or astrocytes in the brain is revived.

13. The method of claim 12, wherein the nanogel enters the microglia and/or astrocytes within the brain such that the PD-L1 antibody is released within the microglia and/or astrocytes.

14. The method of claim 12, wherein the neurodegenerative disorder is Alzheimer's Disease.

15. The method of claim 12, wherein the neurodegenerative disorder comprises Amyotrophic lateral sclerosis, Friedreich's ataxia, Huntington's disease, Lewy body disease, Parkinson's disease, spinal muscular atrophy, multiple sclerosis, neonatal hypoxic-ischemic, stroke, spinal cord injury, brain injury, retina injury, post-traumatic stress disorder, brain tumor, glioblastroma, chemobrain, long COVID, or frontotemporal dementia.

16. The method of claim 12, wherein the first copolymer comprises the reaction production of a third poly(ethylene glycol) with a first pyridine-2-thiol containing monomer and the second copolymer comprises the reaction production of a fourth poly(ethylene glycol) with a second pyridine-2-thiol containing monomer.

17. The method of claim 12, wherein the nAChR targeting ligand comprises a phosphorylcholine group.

18. The method of claim 17, wherein the fourth pendant group comprises the following structure:

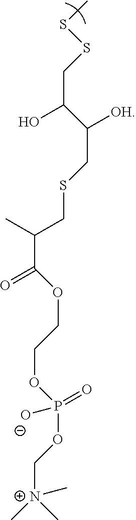 [SMILES: CSSCC(O)C(O)CSCC(C)C(=O)OCCOP(=O)([O-])OC[N+](C)(C)C]

19. The method of claim 12, wherein the RAGE targeting ligand comprises NE-carboxy-methyl-lysine, Nε-carboxy-ethyl-lysine, quinolinic acid, a methylglyoxal-derived hydroimidazolone, an Aβ peptide, HMGB1, or an S100/calgranulins.
